SEQUENCE LISTING (20 sequences)

SEQ ID NO: 1 - protein, 11 aa, Artificial Sequence
  human 19D12/15H12 antibody CDR
RASQSIGSSLH

SEQ ID NO: 2 - protein, 7 aa, Artificial Sequence
  human 19D12/15H12 antibody CDR
YASQSLS

SEQ ID NO: 3 - protein, 9 aa, Artificial Sequence
  human 19D12/15H12 antibody CDR
HQSSRLPHT

SEQ ID NO: 4 - protein, 5 aa, Artificial Sequence
  human 19D12/15H12 antibody CDR
SFAMH

SEQ ID NO: 5 - protein, 10 aa, Artificial Sequence
  human 19D12/15H12 antibody CDR
GFTFSSFAMH

SEQ ID NO: 6 - protein, 16 aa, Artificial Sequence
  human 19D12/15H12 antibody CDR
VIDTRGATYYADSVKG

SEQ ID NO: 7 - protein, 10 aa, Artificial Sequence
  human 19D12/15H12 antibody CDR
LGNFYYGMDV

SEQ ID NO: 8 - protein, 128 aa, Artificial Sequence
  human 19D12/15H12 antibody light chain
MSPSQLIGFLLLWVPASRGEIVLTQVPDFQSVTPKEKVTITCRASQSIGSSLHWYQQKPDQSPKLLIKYASQSLSGVPSRFSGSGSGTDFTLTINSLEAEDAAAYYCHQSSRLPHTFGGGTKVEIKRT

SEQ ID NO: 9 - protein, 128 aa, Artificial Sequence
  modified human 19D12/15H12 antibody light chain-A
MSPSQLIGFLLLWVPASRGEIVLTQSPDSLSVTPGERVTITCRASQSIGSSLHWYQQKPGQSPKLLIYYASQSLSGVPSRFSGSGSGTDFTLTISSLEAEDFAVYYCHQSSRLPHTFGQGTKVEIKRT

SEQ ID NO: 10 - protein, 128 aa, Artificial Sequence
  modified human 19D12/15H12 antibody light chain-B
MSPSQLIGFLLLWVPASRGEIVLTQSPGTLSVSPGERATLSCRASQSIGSSLHWYQQKPGQAPRLLIYYASQSLSGIPDRFSGSGSGTDFTLTISRLEPEDFAVYYCHQSSRLPHTFGQGTKVEIKRT

SEQ ID NO: 11 - protein, 128 aa, Artificial Sequence
  modified human 19D12/15H12 antibody light chain-C
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 12 - protein, 128 aa, Artificial Sequence
  modified human 19D12/15H12 antibody light chain-D
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 13 - protein, 128 aa, Artificial Sequence
  modified human 19D12/15H12 antibody light chain-E
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 14 - protein, 128 aa, Artificial Sequence
  modified human 19D12/15H12 antibody light chain-F
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 15 - protein, 137 aa, Artificial Sequence
  human 19D12/15H12 antibody heavy chain
MEFGLSWVFLVAILKGVQCEVQLVQSGGGLVHPGGSLRLSCAASGFTFSSFAMHWVRQAPGKGLEWISVIDTRGATYYADSVKGRFTISRDNAKNSLYLQMNSLRAEDMAVYYCARLGNFYYGMDVWGQGTTVTVSS

SEQ ID NO: 16 - protein, 137 aa, Artificial Sequence
  modified human 19D12/15H12 antibody heavy chain-A
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 17 - protein, 137 aa, Artificial Sequence
  modified human 19D12/15H12 antibody heavy chain-B
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 18 - DNA, 18 bp, Artificial Sequence
  IGF1R anti-sense nucleic acid
atctctccgcttcctttc

SEQ ID NO: 19 - DNA, 18 bp, Artificial Sequence
  IGF1R anti-sense nucleic acid
atctctccgcttcctttc

SEQ ID NO: 20 - DNA, 18 bp, Artificial Sequence
  IGF1R anti-sense nucleic acid
atctctccgcttcctttc

(19) United States
(12) Patent Application Publication
(10) Pub. No.: US 2006/0286103 A1
(43) Pub. Date: Dec. 21, 2006

(54) Stable antibody formulation

(76) Inventors: Parag Kolhe, Woodbridge, NJ (US); Vinay Radhakrishnan, Thousand Oaks, CA (US); Leonore C. Witchey-Lakshmanan, Piscataway, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/452,167

(22) Filed: Jun. 13, 2006

Related U.S. Application Data
(60) Provisional application No. 60/690,810, filed on Jun. 15, 2005.

(51) Int. Cl. A61K 39/395

(52) U.S. Cl. 424/143.1

(57) ABSTRACT

The present invention provides a pharmaceutical formulation comprising an antibody or antigen-binding fragment thereof that exhibits high stability; along with methods of use thereof.

The present application claims the benefit of U.S. provisional patent application No. 60/690,810; filed Jun. 15, 2005, which is herein incorporated by reference in its entirety.

### FIELD OF THE INVENTION

The present invention provides, inter alia, an pharmaceutical formulation comprising an antibody which exhibits high stability.

### BACKGROUND OF THE INVENTION

Antibodies, like most proteins, must maintain their higher order structure in order to maintain their activity. One problem faced by companies selling antibodies, including therapeutic antibodies, is the identification of conditions under which the antibody can exist for an extended period of time without denaturing and, thus, losing biological activity. In general, therapeutic antibodies on the market are relatively unstable, requiring careful handling and storage at low temperatures. For example, the therapeutic antibodies Avastin™, Herceptin® and Erbitux™ require storage at 2° C. to 8° C. It is likely that the anti-IGF1R antibodies owned by various companies in the industry (e.g., Pfizer, Imclone, Pierre Fabre, Roche and Immunogen) will, similarly, exhibit instability.

The low level of stability exhibited by currently available therapeutic antibodies is disadvantageous due both to the cost and inconvenience presented by the special storage conditions required as well as to the danger of accidental inactivation of the antibody before administration and possible toxicity/immunogenicity due to the degradation/aggregation. There is, thus, a need in the art for a pharmaceutical formulation that will allow therapeutic antibodies, for example anti-IGF1R therapeutic antibodies, to be stable while stored at a wide range of conditions.

### SUMMARY OF THE INVENTION

The present invention addresses the above-referenced need in the art by providing a pharmaceutical formulation, comprising an isolated anti-IGF1R antibody (e.g., monoclonal antibody) or an antigen-binding fragment thereof, that exhibits superior stability and may, thus, be stored at room temperature.

The present invention provides a pharmaceutical formulation comprising a therapeutically effective amount (or, in an embodiment of the invention, any amount) of an isolated antibody (e.g., monoclonal antibody) or an antigen-binding fragment thereof that binds specifically to IGF1R, a buffer such as acetate (e.g., sodium acetate, potassium acetate, magnesium acetate) and acetic acid (e.g., at a concentration of about 1 mM to about 20 mM) and sucrose (e.g., at a concentration of about 5 mg/ml to about 70 mg/ml), optionally, at a pH of about 5.5 to about 6.0 (e.g., 5.5, 5.6. 5.7, 5.8, 5.9, 6.0). In an embodiment, the antibody or fragment comprises one or more light chain complementarity determining regions selected from the group consisting of SEQ ID NOs: 1-3; and one or more heavy chain complementarity determining regions selected from the group consisting of SEQ ID NOs: 4-7.

The present invention also provides a lyophilized pharmaceutical formulation comprising an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; acetate; acetic acid and sucrose at a pH of about 5.5.

The present invention provides a pharmaceutical formulation comprising an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; acetate; acetic acid and sucrose at a pH of about 5.5. In an embodiment of the invention, the formulation is sterile. In an embodiment of the invention the antibody comprises a heavy chain constant region selected from the group consisting of γ1, γ2, γ3 and γ4 or a κ light chain region. In an embodiment of the invention, the formulation is an aqueous solution. In an embodiment of the invention the antibody concentration is about 20 mg/ml. In an embodiment of the invention, the concentration of acetate is about 2.3 mg/ml, the concentration of acetic acid is about 0.18 mg/ml and the concentration of sucrose is about 70 mg/ml. In an embodiment of the invention, the formulation is in association with a further therapeutic agent (e.g., a member selected from the group consisting of: 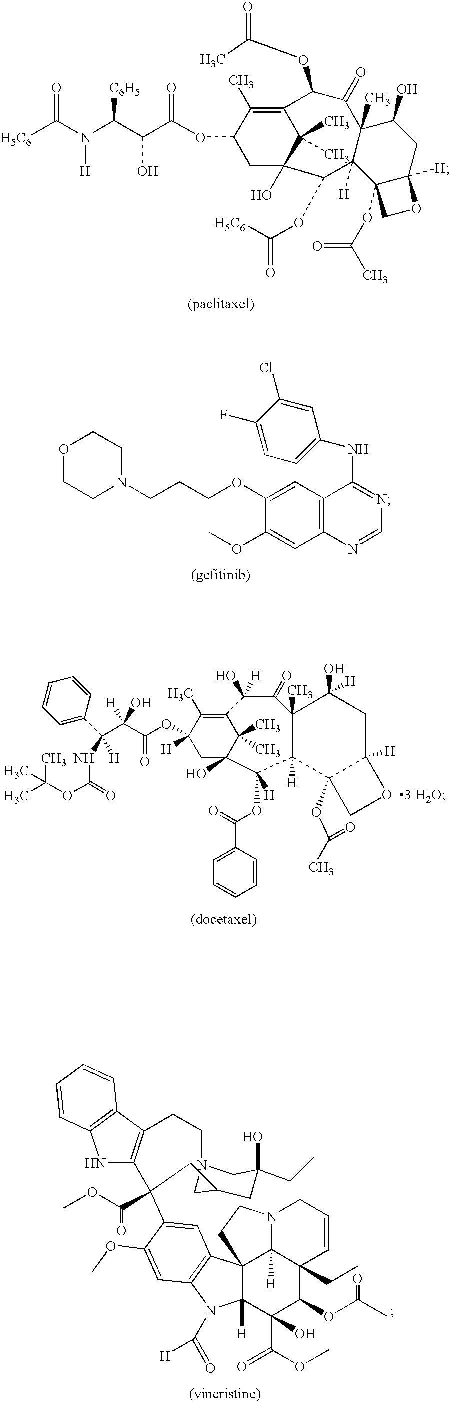 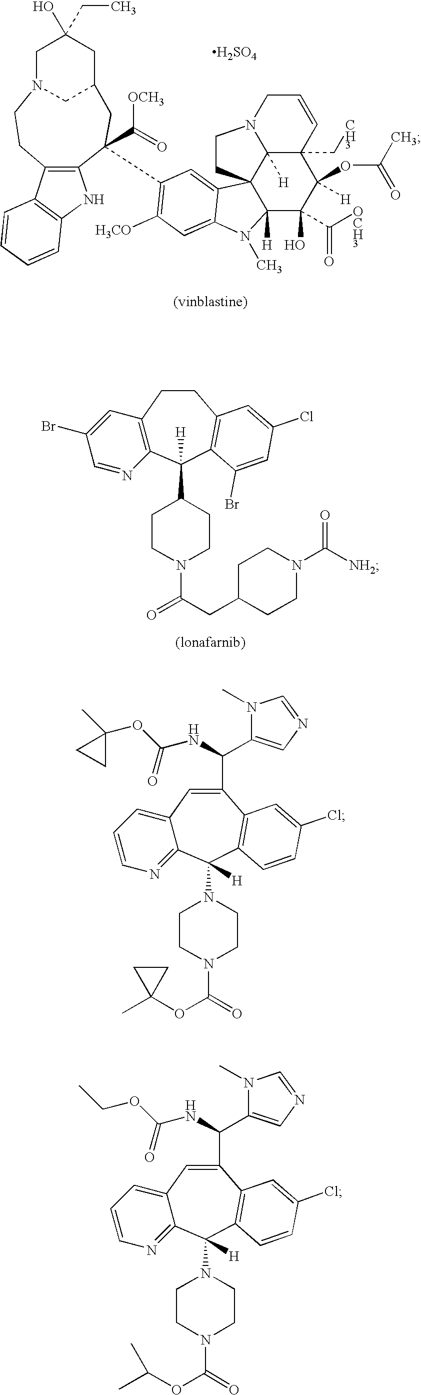 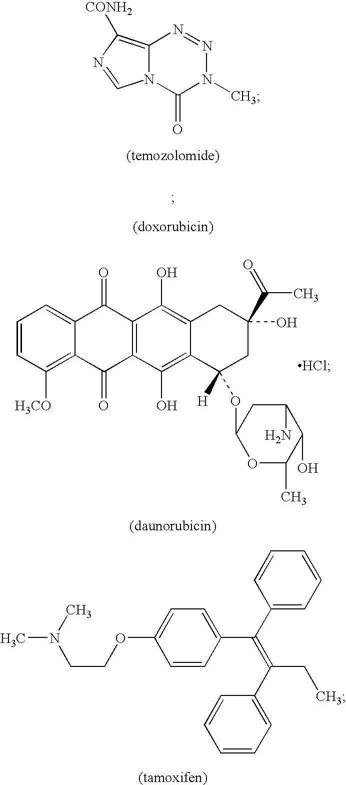 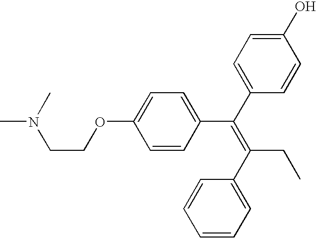
(4-hydroxytamoxifen and any other agent set forth below under “Further therapeutic agents and procedures”). In an embodiment, the formulation comprises, in a single composition, an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; acetate; acetic acid and sucrose at a pH of about 5.5 along with the further therapeutic agent.

The present invention provides, a pharmaceutical formulation, at a pH of 5.5, comprising: (a) 20 mg/ml (or, in an embodiment of the invention, any concentration) of an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17 (e.g., mature LCF and mature HCA); (b) 2.3 mg/ml of sodium acetate trihydrate USP; (c) 0.18 mg/ml of glacial acetic acid USP/Ph. Eur; (d) 70 mg/ml of Sucrose NF, Ph. Eur, BP; and (e) water.

The present invention provides, a lyophilized pharmaceutical formulation, at a pH of 5.5, which, when reconstituted comprises (a) 20 mg/ml (or, in an embodiment of the invention, any concentration) of an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17 (e.g., mature LCF and mature HCA); (by 2.3 mg/ml of sodium acetate trihydrate USP; (c) 0.18 mg/ml of glacial acetic acid USP/Ph. Eur; and (d) 70 mg/ml of Sucrose NF, Ph. Eur, BP.

The present invention provides a vessel (e.g., a glass vial) comprising any of the formulations set forth herein.

The present invention provides an injection device (e.g., hypodermic needle and syringe) comprising any of the formulations set forth herein.

The present invention provides a kit comprising (a) any of the formulations of the invention in a vessel or injection device; and (b) a package insert comprising one or more items of information regarding said formulation selected from the group consisting of pharmacokinetics, pharmacodynamics, clinical studies, efficacy parameters, indications and directions for usage, contraindications, warnings, precautions, adverse reactions, overdosage, proper dosage and administration, how supplied, proper storage conditions, references and patent information.

The present invention also provides a method for treating or preventing a medical disorder mediated by IGF1R, IGF-1 and/or IGF-2, in a subject (e.g., a human), comprising administering, to the subject, a therapeutically effective amount of any of the formulations set forth herein. In an embodiment of the invention, the medical disorder is selected from the group consisting of neuroblastoma, rhabdomyosarcoma, osteosarcoma, any pediatric cancer, acromegaly, ovarian cancer, pancreatic cancer, gastric cancer, benign prostatic hyperplasia, breast cancer, prostate cancer, bone cancer, lung cancer, colorectal cancer, cervical cancer, synovial sarcoma, bladder cancer, Wilm's cancer, ovarian cancer, benign prostatic hyperplasia (BPH), diarrhea associated with metastatic carcinoid and vasoactive intestinal peptide secreting tumors, VIPoma, Werner-Morrison syndrome, kidney cancer, renal cell carcinoma, transitional cell cancer, Ewing Sarcoma, leukemia, acute lymphoblastic leukemia, brain cancer, glioblastoma, non-glioblastoma brain cancer, meningioma, pituitary adenoma, vestibular schwannoma, a primitive neuroectodermal tumor, medulloblastoma, astrocytoma, oligodendroglioma, ependymoma, choroid plexus papilloma, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease. In an embodiment of the invention, the subject is administered a further therapeutic agent in association with the formulation. In an embodiment of the invention, the further therapeutic agent is selected from the group consisting of: 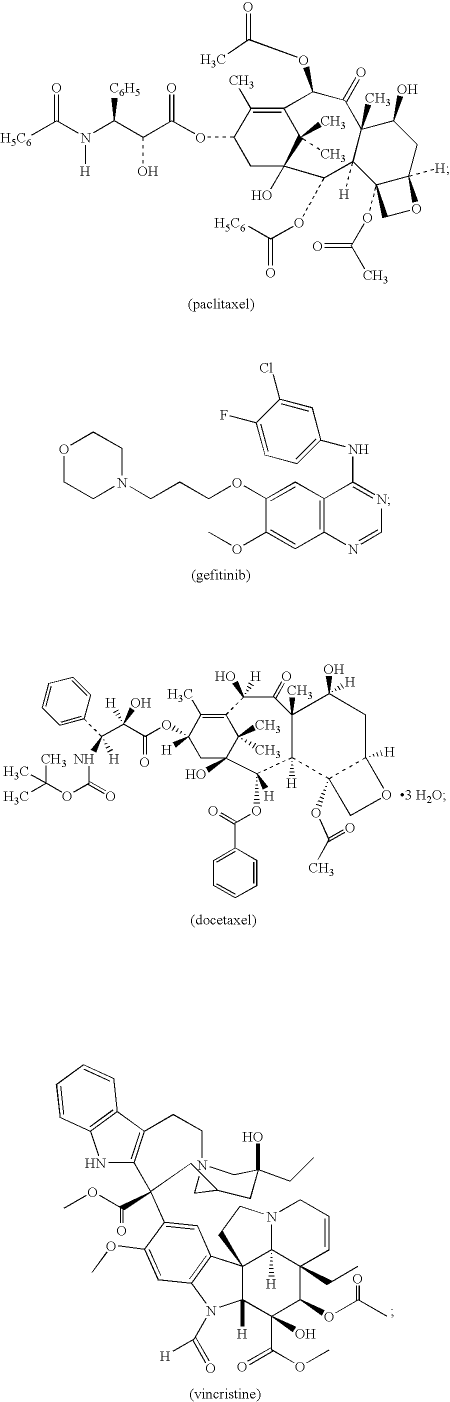 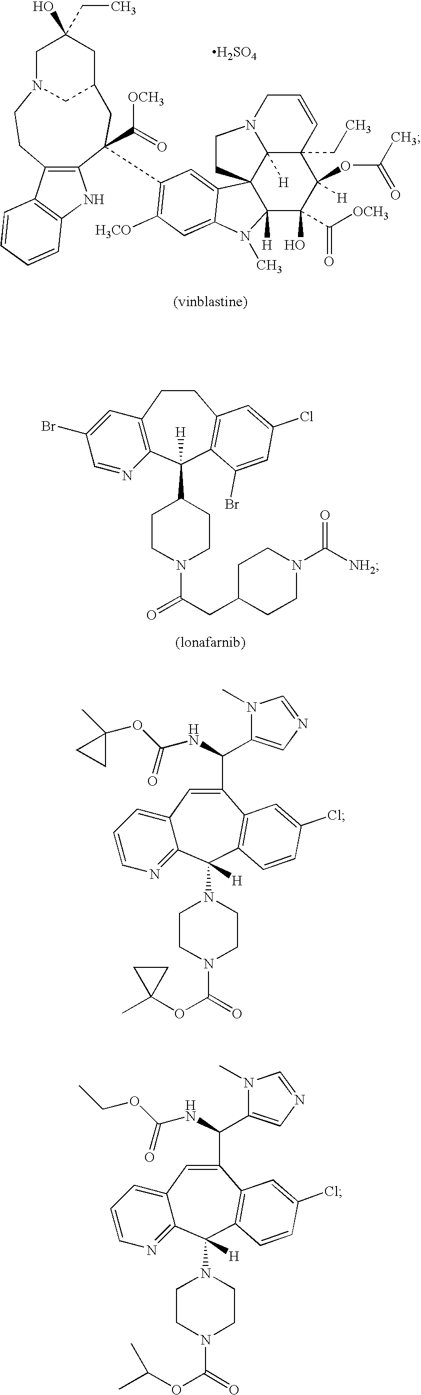 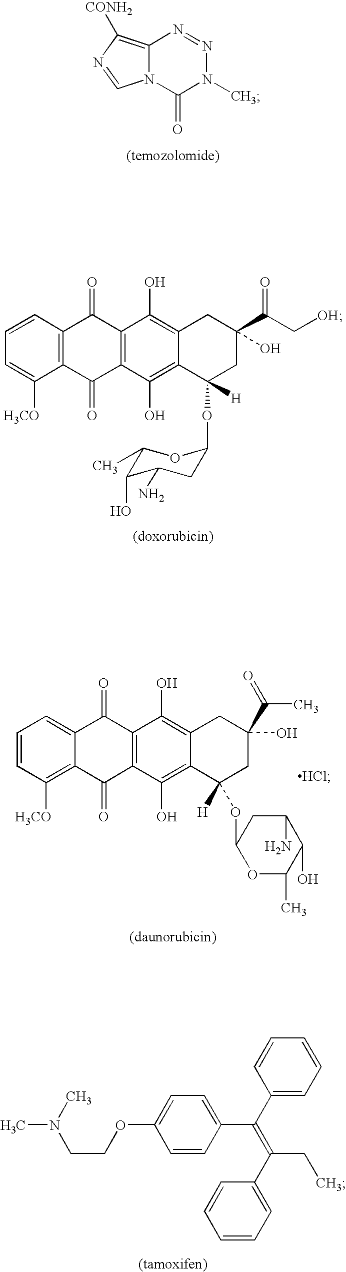 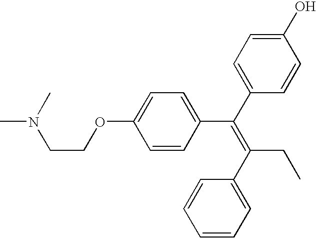
(4-hydroxytamoxifen; and one or more other agents set forth below under “Further therapeutic agents and procedures”). In an embodiment, the formulation is administered to the subject parenterally (e.g., intravenous, intramuscular, intratumoral, intrathecal, intraarterially, subcutaneous). In an embodiment of the invention, the formulation is at pH 5.5 and comprises (a) 20 mg/ml of an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; (b) 2.3 mg/ml of sodium acetate trihydrate USP; (c) 0.18 mg/ml of glacial acetic acid USP/Ph. Eur; (d) 70 mg/ml of Sucrose NF, Ph. Eur, BP; and (e) water.

The present invention provides a method for stabilizing an isolated antibody (e.g., monoclonal antibody) or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; comprising combining said antibody with acetate; acetic acid and sucrose, optionally at a pH of about 5.5. In an embodiment of the invention, the antibody concentration is about 20 mg/ml. In an embodiment of the invention, the concentration of acetate is about 2.3 mg/ml, the concentration of acetic acid is about 0.18 mg/ml and the concentration of sucrose is about 70 mg/ml.

### BRIEF DESCRIPTION OF THE FIGURES

FIG. 1. (a) representative FUV CD scan of anti-IGF1R antibody in acetate buffer of pH 5; (b) representative NUV CD scan of anti-IGF1R antibody in acetate buffer of pH 5.

FIG. 2. (a) Far UV CD Spectrum of anti-IGF1R antibody in various buffers; (b) Change in ellipticity at 217 nm as a function of pH; (c) Change in ellipticity at 235 nm as a function of pH; (d) Change in ellipticity at 235 nm as a function of pH.

FIG. 3. Near UV CD Spectra of anti-IGF1R antibody in various buffers.

FIG. 4. (a) FUV CD Thermal melt data for anti-IGF1R antibody; (b) Tₒₙₛₑₜ (from FUV CD data) as a function of pH.

FIG. 5. (a) NUV CD Thermal melt data for anti-IGF1R antibody; (b) Tₒₙₛₑₜ (from NUV CD data) as a function of pH.

FIG. 6. (a) DSC thermograms for anti-IGF1R antibody; (b) Tₒₙₛₑₜ (from DSC data) as a function of pH; (c) Tₘ₁ (from DSC data) as a function of pH.

FIG. 7. (a) Particle size distribution of anti-IGF1R antibody; (b) Change in size distribution of anti-IGF1R antibody (in phosphate buffer of pH 7) at various temperatures.

FIG. 8. (a) Tₒₙₛₑₜ of aggregation data for anti-IGF1R antibody; (b) Tₒₙₛₑₜ of aggregation as a function of pH.

FIG. 9. (Tₒₙₛₑₜ from FUV CD data): Effect of Sodium Chloride on Tₒₙₛₑₜ.

FIG. 10. (Tₒₙₛₑₜ from FUV CD data): Effect of Sucrose on Tₒₙₛₑₜ.

FIG. 11. Stability of the anti-IGF1R antibody in acetate buffer at pH 5.5 with 7% w/v sucrose.

### DETAILED DESCRIPTION OF THE INVENTION

Antibodies in the formulation of the present invention exhibit superior stability. The formulations of the invention allow antibodies contained in them to remain intact even after several months of storage at room temperature (e.g., 25° C.). Such high stability makes the formulations of the invention particularly useful, for example, because the formulations allow the clinician, patient or pharmacy possessing the formulation to choose conveniently between storage at room temperature or under refrigeration. Moreover, the high stability ensures that the antibodies retain their biological activity over time which, in turn, ensures that they retain their efficacy e.g., when used to treat a cancerous condition. The particular benefits of the formulations of the invention can be realized even in the absence of storage at room temperature (e.g., under refrigeration at 4° C.). When stored at 4° C., the formulations exhibit somewhat greater stability.

The present invention provides, inter alia, a pharmaceutical formulation comprising any anti-IGF1R antibody, a buffer such as acetate/acetic acid buffer and sucrose at about pH 5.5 to about 6.0 (e.g., 5.5., 5.6, 5.7, 5.8, 5.9, 6.0; in an embodiment of the invention, pH is about 5.3 or 5.4). The formulation of the present invention is useful, for example, for administration to a patient for the treatment or prevention of any medical disorder mediated by elevated expression or activity of IGF1R or by elevated expression of its ligand (e.g., IGF-I or IGF-II) and which may be treated or prevented by modulation of IGF1R ligand binding, activity or expression. In an embodiment of the invention, the disease or condition is mediated by an increased level of IGF1R, IGF-I or IGF-II and is treated or prevented by decreasing IGF1R ligand binding, activity (e.g., autophosphorylation activity) or expression.

In an embodiment of the invention, the formulation of the invention is as set forth below:

In an embodiment of the invention, the formulation of the invention is as set forth below:

| Ingredient | mg/mL |
|---|---|
| Anti IGF1R antibody (API) | 20.0 |
| Sodium Acetate Trihydrate USP | 2.30 |
| Glacial Acetic Acid USP/Ph. Eur | 0.18 |
| Sucrose NF, Ph. Eur, BP | 70.0 |
| Water for Injection USP, Ph. Eur. | q.s. ad 1 mL |

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, N.Y., Lieberman, et al., (eds.) (1990) Pharmaceutical Dosage Forms: Tablets Dekker, N.Y.; and Lieberman, et al., (eds.) (1990), Pharmaceutical Dosage Forms: Disperse Systems Dekker, N.Y., Kenneth A. Walters (ed.) (2002) Dermatological and Transdermal Formulations (Drugs and the Pharmaceutical Sciences), Vol 119, Marcel Dekker.

The term “subject” or “patient” includes any organism, for example, a mammal (e.g., rat, mouse, cat, dog, horse, rabbit, monkey, ape, primate, chimpanzee, bird or cow) such as a human including pediatric (e.g., 1, 2, 3, 4, 5, 6, 7, 8, 9, 10, 11, 12, 13, 14, 15, 16, 17 or 18 years of age) and geriatric subjects (e.g., 60, 65, 70, 75, 80, 85, 90 or more years of age) thereof.

### Antibodies

The present invention comprises a pharmaceutical composition comprising an anti-IGF1R antibody or antigen-binding fragment thereof. The term “anti-IGF1R” antibody includes any antibody comprising e.g., 15H12/19D12 HC (heavy chain), HCA or HCB and/or 15H12/19D12 LC (light chain), LCA, LCB, LCC, LCD, LCE or LCF (or any mature fragment thereof) (e.g., LCF and HCA). An anti-IGF1R antibody or antigen-binding fragment thereof includes, in an embodiment of the invention, antibodies and fragments that bind specifically to IGF1R or any fragment thereof (e.g., sIGF1R). Antibodies include, in an embodiment of the invention, monoclonal antibodies, polyclonal antibodies, humanized antibodies, chimeric antibodies, anti-idiotypic antibodies and bispecific antibodies and fragments include Fab antibody fragments, F(ab)₂ antibody fragments, Fv antibody fragments (e.g., V_{H} or V_{L}), single chain Fv antibody fragments and dsFv antibody fragments. Furthermore, the anti-IGF1R antibodies of the invention, in an embodiment of the invention, are fully human antibodies. In an embodiment of the invention, the anti-IGF1R antibody is a monoclonal, fully human antibody. In an embodiment of the invention, the anti-IGF1R antibody includes one or more of the variable regions and/or CDRs whose amino acid and nucleotide sequences are set forth herein:

| | |
|---|---|
| RASQSIGSSLH; | (SEQ ID NO:1) |
| YASQSLS; | (SEQ ID NO:2) |
| HQSSRLPHT; | (SEQ ID NO:3) |
| SFAMH (SEQ ID NO:4) | |
| GFTFSSFAMH; | (SEQ ID NO:5) |
| VIDTRGATYYADSVKG; | (SEQ ID NO:6) |
| LGNFYYGMDV; | (SEQ ID NO:7) |

The scope of the present invention includes a pharmaceutical formulation comprising an anti-IGF1R antibody comprising a light chain variable region linked to a constant region, for example, a κ chain and/or a heavy chain variable region linked to a constant region, for example a γ1, γ2, γ3 or γ4 constant region.

In an embodiment of the invention, the anti-IGF1R antibodies of the invention recognize human IGF1R, and/or sIGF1R (any soluble fragment of IGF1R); however, the present invention includes antibodies that recognize IGF1R from different species, for example, mammals (e.g., mouse, rat, rabbit, sheep or dog).

In an embodiment of the invention, an antibody or antigen-binding fragment thereof that binds “specifically” to IGF1R (e.g., human IGF1R) binds with a Kd of about 10⁻⁸ M or 10⁻⁷ M or a lower number; or, in an embodiment of the invention, with a Kd of about 1.28×10⁻¹ M or a lower number by Biacore measurement or with a Kd of about 2.05×10⁻¹² or a lower number by KinExA measurement. In another embodiment of the invention, an antibody or antigen-binding fragment thereof that binds “specifically” to human IGF1R binds exclusively to human IGF1R and to no other protein at significant levels.

In an embodiment, an anti-IGF1R antibody of the invention, particularly an anti-IGF1R antibody that binds “specifically” to IGF1R, comprises one or more of the following characteristics:
(a) Binds to IGF1R with a K_{d} of about 86×10⁻¹¹ or a lower number;
(b) Has an off rate (K_{off}) for IGF1R of about 6.50×10⁻⁵ or a lower number;
(c) Has an on rate (Kₒₙ) for IGF1R of about 0.7×10⁵ or a higher number;
(d) Competes with IGF1 for binding to IGF1R;
(e) Inhibits autophosphorylation of IGF1R; and
(f) Inhibits anchorage-independent growth of a cell expressing IGF1R.

“K_{off}” refers to the off-rate constant for dissociation of the antibody from an antibody/antigen complex.

“Kₒₙ” refers to the rate at which the antibody associates with the antigen.

“K_{d}” refers to the dissociation constant of a particular antibody/antigen interaction. K_{d}=K_{off}/Kₒₙ.

The term “monoclonal antibody,” as used herein, includes an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method first described by Kohler, et al., (1975) Nature 256: 495 or other methods known in the art.

A polyclonal antibody is an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

The term “fully human antibody” refers to an antibody which comprises human immunoglobulin amino acid sequences only. A fully human antibody may contain murine carbohydrate chains if produced in a mouse, in a mouse cell or in a hybridoma derived from a mouse cell. Similarly, “mouse antibody” refers to an antibody which comprises mouse immunoglobulin sequences only.

The present invention includes “chimeric antibodies”—an antibody which comprises a variable region of one species fused or chimerized with an antibody region (e.g., constant region) from another species (e.g., mouse, horse, rabbit, dog, cow, chicken). These antibodies may be used to modulate the expression or activity of IGF1R in the non-human species.

“Single-chain Fv” or “sFv” antibody fragments have the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

“Disulfide stabilized Fv fragments” and “dsFv” refer to antibody molecules comprising a variable heavy chain (V_{H}) and a variable light chain (V_{L}) which are linked by a disulfide bridge.

Antibody fragments for use in the formulations within the scope of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}—C_{L} chain appended to a V_{H}—C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

An Fv fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. [gG-1, IgG-2, IgG-3 and IgG-4; IgA-1 and IgA-2.

The anti-IGF1R antibodies of the formulations of the invention may also be conjugated to a chemical moiety. The chemical moiety may be, inter alia, a polymer, a radionuclide or a cytotoxic factor. In an embodiment of the invention, the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments of the formulations of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments of the formulations of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments of the formulations of the present invention can also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies and antibody fragments of the formulations of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In an embodiment, 15H12/19D12 LC, LCA, LCB, LCC, LCD, LCE or LCF is dimerized with any other immunoglobulin heavy chain, for example, any immunoglobulin heavy chain set forth herein. Likewise, in an embodiment,15H12/19D12 HC, HCA or HCB is dimerized with any light chain, for example, any light chain set forth herein. For example, 15H12/19D12 HCA or HCB can be dimerized with 15H12/19D12 LCC, LCD, LCE or LCF. In an embodiment, the light immunoglobulin chain and or the heavy immunoglobulin chain of an anti-IGF1R antibody of the invention is a mature chain.

Antibody chains are shown below. Dotted underscored type encodes the signal peptide. Solid underscored type encodes the CDRs. Plain type encodes the framework regions. Antibody chains are mature fragments which lack the signal peptide.

19D12/15H12 Light Chain (SEQ ID NO: 8)

Met Ser Pro Ser Gln Leu Ile Gly Phe Leu Leu Leu
Trp Val Pro Ala Ser Arg Gly Glu Ile Val Leu Thr
Gln Val Pro Asp Phe Gln Ser Val Thr Pro Lys Glu
Lys Val Thr Ile Thr Cys Arg Ala Ser Gln Ser Ile
Gly Ser Ser Leu His Trp Tyr Gln Gln Lys Pro Asp
Gln Ser Pro Lys Leu Leu Ile Lys Tyr Ala Ser Gln
Ser Leu Ser Gly Val Pro Ser Arg Phe Ser Gly Ser
Gly Ser Gly Thr Asp Phe Thr Leu Thr Ile Asn Ser
Leu Glu Ala Glu Asp Ala Ala Ala Tyr Tyr Cys His
Gln Ser Ser Arg Leu Pro His Thr Phe Gly Gly Gly
Thr Lys Val Glu Ile Lys Arg Thr

19D12/15H12 Light Chain-A (SEQ ID NO: 9)

Met Ser Pro Ser Gln Leu Ile Gly Phe Leu Leu Leu
Trp Val Pro Ala Ser Arg Gly Glu Ile Val Leu Thr
Gln Ser Pro Asp Ser Leu Ser Val Thr Pro Gly Glu
Arg Val Thr Ile Thr Cys Arg Ala Ser Gln Ser Ile
Gly Ser Ser Leu His Trp Tyr Gln Gln Lys Pro Gly
Gln Ser Pro Lys Leu Leu Ile Tyr Tyr Ala Ser Gln
Ser Leu Ser Gly Val Pro Ser Arg Phe Ser Gly Ser
Gly Ser Gly Thr Asp Phe Thr Leu Thr Ile Ser Ser
Leu Glu Ala Glu Asp Phe Ala Val Tyr Tyr Cys His
Gln Ser Ser Arg Leu Pro His Thr Phe Gly Gln Gly
Thr Lys Val Glu Ile Lys Arg Thr

19D12/15H12 Light Chain-B (SEQ ID NO: 10)

Met Ser Pro Ser Gln Leu Ile Gly Phe Leu Leu Leu
Trp Val Pro Ala Ser Arg Gly Glu Ile Val Leu Thr
Gln Ser Pro Gly Thr Leu Ser Val Ser Pro Gly Glu
Arg Ala Thr Leu Ser Cys Arg Ala Ser Gln Ser Ile
Gly Ser Ser Leu His Trp Tyr Gln Gln Lys Pro Gly
Gln Ala Pro Arg Leu Leu Ile Tyr Tyr Ala Ser Gln
Ser Leu Ser Gly Ile Pro Asp Arg Phe Ser Gly Ser
Gly Ser Gly Thr Asp Phe Thr Leu Thr Ile Ser Arg
Leu Glu Pro Glu Asp Phe Ala Val Tyr Tyr Cys His
Gln Ser Ser Arg Leu Pro His Thr Phe Gly Gln Gly
Thr Lys Val Glu Ile Lys Arg Thr

19D12/15H12 Light Chain-C (SEQ ID NO: 11)

M S P S Q L I G F L L L W V P A S
R G E I V L T Q S P D S L S V T P
G E R V T I T C R A S Q S I G S S
L H W Y Q Q K P G Q S P K L L I K
Y A S Q S L S G V P S R F S G S G
S G T D F T L T I S S L E A E D A
A A Y Y C H Q S S R L P H T F G Q
G T K V E I K R T

Modified 19D12/15H12 Light Chain-D (SEQ ID NO: 12)

M S P S Q L I G F L L L W V P A S
R G E I V L T Q S P D S L S V T P
G E R V T I T C R A S Q S I G S S
L H W Y Q Q K P G Q A P K L L I K
Y A S Q S L S G V P S R F S G S G
S G T D F T L T I S S L E A E D F
A V Y Y C H Q S S R L P H T F G Q
G T K V E I K R T

Modified 19D12/15H12 Light Chain-E (SEQ ID NO: 13)

M S P S Q L I G F L L L W V P A S
R G E I V L T Q S P G T L S V S P
G E R A T L S C R A S Q S I G S S
L H W Y Q Q K P G Q A P R L L I K
Y A S Q S L S G I P D R F S G S G
S G T D F T L T I S R L E P E D A
A A Y Y C H Q S S R L P H T F G Q
G T K V E I K R T

19D12/15H12 Light Chain-F (SEQ ID NO: 14)

M S P S Q L I G F L L L W V P A S
R G S I V L T Q S P G T L S V S P
G E R A T L S C R A S Q S I G S S
L H W Y Q Q K P G Q A P R L L I K
Y A S Q S L S G I P D R F S G S G
S G T D F T L T I S R L E P E D F
A V Y Y C H Q S S R L P H T F G Q
G T K V E I K H T

19D12/15H12 heavy chain (SEQ ID NO: 15)

Met Glu Phe Gly Leu Ser Trp Val Phe Leu Val Ala
Ile Leu Lys Gly Val Gln Cys Glu Val Gln Leu Val
Gln Ser Gly Gly Gly Leu Val His Pro Gly Gly Ser
Leu Arg Leu Ser Cys Ala Ala Ser Gly Phe Thr Phe
Ser Ser Phe Ala Met His Trp Val Arg Gln Ala Pro
Gly Lys Gly Leu Glu Trp Ile Ser Val Ile Asp Thr
Arg Gly Ala Thr Tyr Tyr Ala Asp Ser Val Lys Gly
Arg Phe Thr Ile Ser Arg Asp Asn Ala Lys Asn Ser
Leu Tyr Leu Gln Met Asn Ser Leu Arg Ala Glu Asp
Met Ala Val Tyr Tyr Cys Ala Arg Leu Gly Asn Phe
Tyr Tyr Gly Met Asp Val Trp Gly Gln Gly Thr Thr
Val Thr Val Ser Ser

19D12/15H12 heavy chain-A (SEQ ID NO: 16)

Met Glu Phe Gly Leu Ser Trp Val Phe Leu Val Ala
Ile Leu Lys Gly Val Gln Cys Glu Val Gln Leu Val
Gln Ser Gly Gly Gly Leu Val Lys Pro Gly Gly Ser
Leu Arg Leu Ser Cys Ala Ala Ser Gly Phe Thr Phe
Ser Ser Phe Ala Met His Trp Val Arg Gln Ala Pro
Gly Lys Gly Leu Glu Trp Ile Ser Val Ile Asp Thr
Arg Gly Ala Thr Tyr Tyr Ala Asp Ser Val Lys Gly
Arg Phe Thr Ile Ser Arg Asp Asn Ala Lys Asn Ser
Leu Tyr Leu Gln Met Asn Ser Leu Arg Ala Glu Asp
Thr Ala Val Tyr Tyr Cys Ala Arg Leu Gly Asn Phe
Tyr Tyr Gly Met Asp Val Trp Gly Gln Gly Thr Thr
Val Thr Val Ser Ser

Modified 19D12/15H12 heavy chain-B (SEQ ID NO: 17)

Met Glu Phe Gly Leu Ser Trp Val Phe Leu Val Ala
Ile Leu Lys Gly Val Gln Cys Glu Val Gln Leu Val
Gln Ser Gly Gly Gly Leu Val Gln Pro Gly Gly Ser
Leu Arg Leu Ser Cys Ala Ala Ser Gly Phe Thr Phe
Ser Ser Phe Ala Met His Trp Val Arg Gln Ala Pro
Gly Lys Gly Leu Glu Trp Ile Ser Val Ile Asp Thr
Arg Gly Ala Thr Tyr Tyr Ala Asp Ser Val Lys Gly
Arg Phe Thr Ile Ser Arg Asp Asn Ala Lys Asn Ser
Leu Tyr Leu Gln Met Asn Ser Leu Arg Ala Glu Asp
Thr Ala Val Tyr Tyr Cys Ala Arg Leu Gly Asn Phe
Tyr Tyr Gly Met Asp Val Trp Gly Gln Gly Thr Thr
Val Thr Val Ser Ser

Cell lines containing plasmids encoding the above-referenced antibody chains were deposited at the American Type Culture Collection as follows:
(i) CMV promoter-15H12/19D12 HCA (γ4)−

Deposit name: “15H12/19D12 HCA (γ4)”;

ATCC accession No.: PTA-5214;
(ii) CMV promoter-15H12/19D12 HCB (γ4)−

Deposit name: “15H12/19D12 HCB (γ4)”;

ATCC accession No.: PTA-5215;
(iii) CMV promoter-15H12/19D12 HCA (γ1)−

Deposit name: “15H12/19D12 HCA (γ1)”;

ATCC accession No.: PTA-5216;
(iv) CMV promoter-15H12/19D12 LCC (κ)−

Deposit name: “15H12/19D12 LCC (κ)”;

ATCC accession No.: PTA-5217;
(v) CMV promoter-15H12/19D12 LCD (κ)−

Deposit name: “15H12/19D12 LCD (κ)”;

ATCC accession No.: PTA-5218;
(vi) CMV promoter-15H12/19D12 LCE (κ)−

Deposit name: “15H12/19D12 LCE (κ)”;

ATCC accession No.: PTA-5219; and
(vii) CMV promoter-15H12/19D12 LCF (κ)−

Deposit name: “15H12/19D12 LCF (κ)”;

ATCC accession No.: PTA-5220;

HCA is heavy chain A; HCB is heavy chain B, LCC is light chain C; LCD is light chain D; LCE is light chain E and LCF is light chain F.

The above-identified plasmids were deposited, under the Budapest Treaty, on May 21, 2003 with the American Type Culture Collection (ATCC); 10801 University Boulevard; Manassas, Va. 20110-2209. All restrictions on access to the plasmids deposited in ATCC will be removed upon grant of a patent (see published U.S. patent application no. US2004/0018191).

The present application comprises formulations and methods of use thereof, as set forth herein, comprising antibodies and antigen-binding fragments thereof whose immunoglobulin chains (e.g., mature chains thereof), for example, heavy chains or light chains, which are encoded by the inserts in the plasmids in the cell lines deposited at the ATCC as described above. Formulations comprising immunoglobulins encoded by the plasmids comprising a different constant region than that indicated above are also within the scope of the present invention along with methods of use thereof e.g., as set forth herein.

### Further Therapeutic Agents and Procedures

In an embodiment of the invention, a further chemotherapeutic agent is provided and/or administered in association with the anti-IGF1R formulation of the invention. In an embodiment, the further chemotherapeutic agent is a platinum-based compound, a signal transduction inhibitor, a cell cycle inhibitor, a IGF/IGF1R system modulator (e.g., inhibitors or activators), a farnesyl protein transferase (FPT) inhibitor, an epidermal growth factor receptor (EGFR) inhibitor, a HER2 inhibitor, a vascular epidermal growth factor (VEGF) receptor inhibitor, a mitogen activated protein (MAP) kinase inhibitor, a MEK inhibitor, an AKT inhibitor, a mTOR inhibitor, a pl3 kinase inhibitor, a Raf inhibitor, a cyclin dependent kinase (CDK) inhibitor, a microtubule stabilizer, a microtubule inhibitor, a SERMs/Antiestrogen, an aromatase inhibitor, an anthracycline, a proteasome inhibitor or an agent which inhibits insulin-like growth factor (IGF) production.

Compositions and methods of the invention include an anti-IGF1R formulation “in association with” one or more further therapeutic agents or procedures. The term “in association with” indicates that the components (e.g., anti-IGF1R antibody along with paclitaxel) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody formulation is administered parenterally and gefitinib is administered orally).

In an embodiment of the invention, the anti-IGF1R formulation of the invention is provided and/or administered in association with a farnesyl protein transferase (FPT) inhibitor including tricyclic amide compounds such as any of those disclosed in U.S. Pat. No. 5,719,148 or in U.S. Pat. No. 5,874,442. In an embodiment, the anti-IGF1R formulation of the invention is provided in association with any compound represented by the following formula: 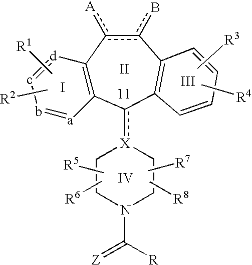
or a pharmaceutically acceptable salt or solvate thereof, wherein: one of a, b, c and d represents N or NR⁹ wherein R⁹ is O—, —CH₃ or —(CH₂)ₙCO₂H wherein n is 1 to 3, and the remaining a, b, c and d groups represent CR¹ or CR²; or each of a, b, c, and d are independently selected from CR¹ or CR²; each R¹ and each R² is independently selected from H, halo, —CF₃, —OR¹⁰, —COR¹⁰, —SR¹⁰, —S(O)ₜR¹¹ (wherein t is 0, 1 or 2), —SCN, —N(R¹⁰)₂, —NO₂, —OC(O)R¹⁰, —CO₂R¹⁰, —OCO₂R¹¹, —CN, —NHC(O)R¹⁰, —NHSO₂R¹⁰, —CONHR¹⁰, —CONHCH₂CH₂OH, —NR¹⁰COOR¹¹, —SR¹¹C(O)OR¹¹, 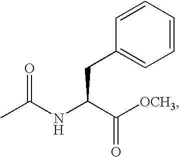
—SR¹¹N(R⁷⁵)₂ (wherein each R⁷⁵ is independently selected from H and —C(O)OR¹¹), benzotriazol-1-yloxy, tetrazol-5-ylthio, or substituted tetrazol-5-ylthio, alkynyl, alkenyl or alkyl, said alkyl or alkenyl group optionally being substituted with halo, —OR¹⁰ or —CO₂R¹⁰; R³ and R⁴ are the same or different and each independently represents H, any of the substituents of R¹ and R², or R³ and R⁴ taken together represent a saturated or unsaturated C₅-C₇ fused ring to the benzene ring; R⁵, R⁶, R⁷ and R⁸ each independently represents H, —CF₃, —COR¹⁰, alkyl or aryl, said alkyl or aryl optionally being substituted with —OR¹⁰, —SR¹⁰, —S(O)ₜR¹¹, —NR¹⁰COOR¹¹, —N(R¹⁰)₂, —NO₂, —COR¹⁰, —OCOR¹⁰, —OCO₂R¹¹, —CO₂R¹⁰, OPO₃R¹⁰ or one of R⁵, R⁶, R⁷ and R⁸ can be taken in combination with R⁴⁰ as defined below to represent —(CH₂)ᵣ— wherein r is 1 to 4 which can be substituted with lower alkyl, lower alkoxy, —CF₃ or aryl, or R⁵ is combined with R⁶ to represent ═O or ═S and/or R⁷ is combined with R⁸ to represent ═O or ═S; R¹⁰ represents H, alkyl, aryl, or aralkyl; R¹¹ represents alkyl or aryl; X represents N, CH or C, which C may contain an optional double bond, represented by the dotted line, to carbon atom 11; the dotted line between carbon atoms 5 and 6 represents an optional double bond, such that when a double bond is present, A and B independently represent —R¹⁰, halo, —OR¹¹, —OCO₂R¹¹ or —OC(O)R¹⁰ and when no double bond is present between carbon atoms 5 and 6, A and B each independently represent H₂, —(OR¹¹)₂; H and halo, dihalo, alkyl and H, (alkyl)₂, —H and —OC(O)R¹⁰, H and —OR¹⁰, ═O, aryl and H, ═NOR¹⁰ or —O—(CH₂)ₚ—O— wherein p is 2, 3 or 4; R represents R⁴⁰, R⁴², R⁴⁴, or R⁵⁴, as defined below; R⁴⁰ represents H, aryl, alkyl, cycloalkyl, alkenyl, alkynyl or -D wherein -D represents 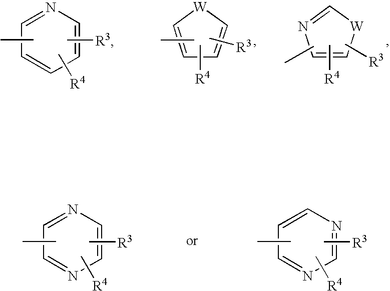
wherein R³ and R⁴ are as previously defined and W is O, S or NR¹⁰ wherein R¹⁰ is as defined above; said R⁴⁰ cycloalkyl, alkenyl and alkynyl groups being optionally substituted with from 1-3 groups selected from halo, —CON(R¹⁰)₂, aryl, —CO₂R¹⁰, —OR¹², —SR¹², —N(R¹⁰)₂, —N(R¹⁰)CO₂R¹¹, —COR¹², —NO₂ or D, wherein -D, R¹⁰ and R¹¹ are as defined above and R¹² represents R¹⁰, —(CH₂)ₘOR¹⁰ or —(CH₂)_{q}CO₂R¹⁰ wherein R¹⁰ is as previously defined, m is 1 to 4 and q is 0 to 4; said alkenyl and alkynyl R⁴⁰ groups not containing —OH, —SH or —N(R¹⁰)₂ on a carbon containing a double or triple bond respectively; or R⁴⁰ represents phenyl substituted with a group selected from —SO₂NH₂, —NHSO₂CH₃, —SO₂NHCH₃, —SO₂CH₃, —SOCH₃, —SCH₃, or —NHSO₂CF₃, preferably, said group is located in the para position of the phenyl ring; or R⁴⁰ represents a group selected from

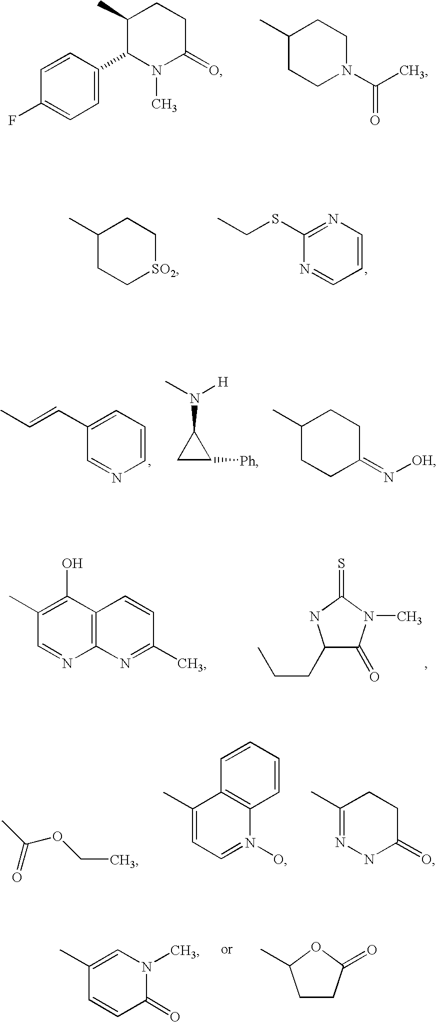

R⁴² represents 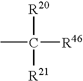
wherein R²⁰, R²¹ and R⁴⁶ are each independently selected from the group consisting of:

(1) H;

(2) —(CH₂)_{q}SC(O)CH₃ wherein q is 1 to 3;

(3) —(CH₂)_{q}OSO₂CH₃ wherein q is 1 to 3;

(4) —OH;

(5) —CS(CH₂)_{w}(substituted phenyl) wherein w is 1 to 3 and the substitutents on said substituted phenyl group are the same substitutents as described below for said substituted phenyl;

(6) —NH₂;

(7) —NHCBZ;

(8) —NHC(O)OR²² wherein R²² is an alkyl group having from 1 to 5 carbon atoms, or R²² represents phenyl substituted with 1 to 3 alkyl groups;

(9) alkyl;

(10) —(CH₂)ₖphenyl wherein k is 1 to 6;

(11) phenyl;

(12) substituted phenyl wherein the substituents are selected from the group consisting of: halo, NO₂, —OH, —OCH₃, —N H₂, —NHR²², —N(R²²)₂, alkyl, —O(CH₂)ₜphenyl (wherein t is from 1 to 3), and —O(CH₂)tsubstituted phenyl (wherein t is from 1 to 3);

(13) naphthyl;

(14) substituted naphthyl, wherein the substituents are as defined for substituted phenyl above;

(15) bridged polycyclic hydrocarbons having from 5 to 10 carbon atoms;

(16) cycloalkyl having from 5 to 7 carbon atoms;

(17) heteroaryl;

(18) hydroxyalkyl;

(19) substituted pyridyl or substituted pyridyl N-oxide wherein the substituents are selected from methylpyridyl, morpholinyl, imidazolyl, 1-piperidinyl, 1-(4-methylpiperazinyl), —S(O)ₜR¹¹, or any of the substituents given above for said substituted phenyl, and said substitutents are bound to a ring carbon by replacement of the hydrogen bound to said carbon;

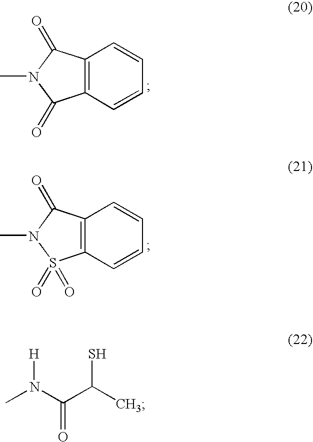

(23) —NHC(O)—(CH₂)ₖ-phenyl or —NH(O)—(CH₂)ₖ-substitued phenyl, wherein said k is as defined above;

(24) piperidine Ring V: 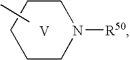
wherein R⁵⁰ represents H, alkyl, alkylcarbonyl, alkyloxycarbonyl, haloalkyl, or —C(O)NH(R¹⁰) wherein R¹⁰ is H or alkyl; ; Ring V includes 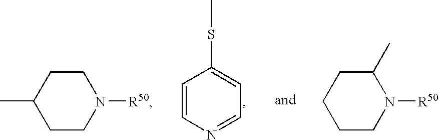
examples of Ring V include:

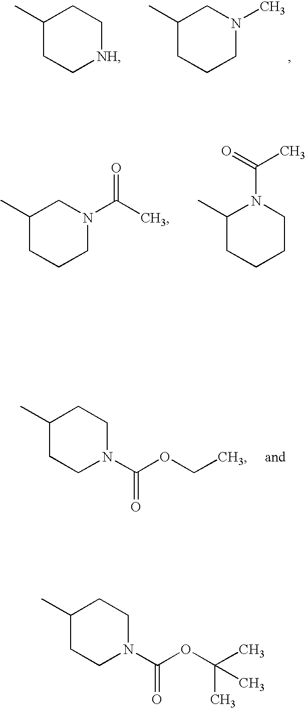

(25) —NHC(O)CH₂C₆H₅ or —NHC(O)CH₂-substituted-C₆H₅;

(26) —NHC(O)OC₆H₅;

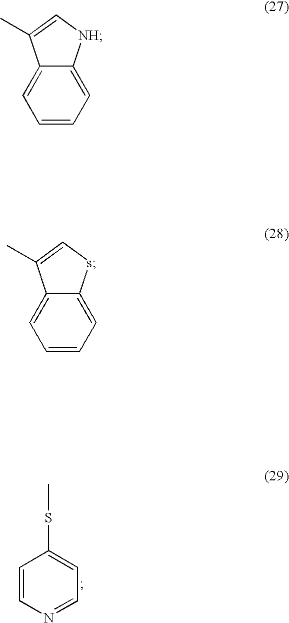

(30) —OC(O)-heteroaryl, for example

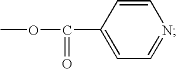

(31) —O-alkyl (e.g., —OCH₃); and

(32) —CF₃;

(33) —CN;

(34) a heterocycloalkyl group of the formula

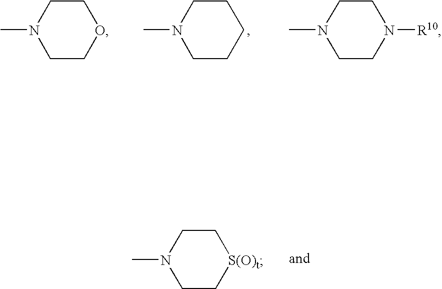

(35) a piperidinyl group of the formula 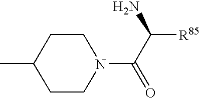
wherein R⁸⁵ is H, alkyl, or alkyl substituted by —OH or —SCH₃; or R²⁰ and R²¹ taken together form a ═O group and the remaining R⁴⁶ is as defined above; or

Two of R²⁰, R²¹ and R⁴⁶ taken together form piperidine Ring V 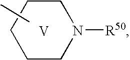
wherein R50 represents H, alkyl (e.g., methyl), alkylcarbonyl (e.g., CH3C(O)—), alkyloxycarbonyl (e.g., —C(O)O-t-C4H9, -C(O)OC2H5, and —C(O)OCH3), haloalkyl (e.g., trifluro-methyl), or —C(O)NH(R10) wherein R10 is H or alkyl; Ring V includes 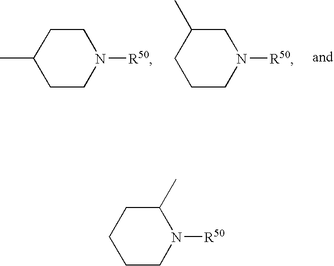
examples of Ring V include: 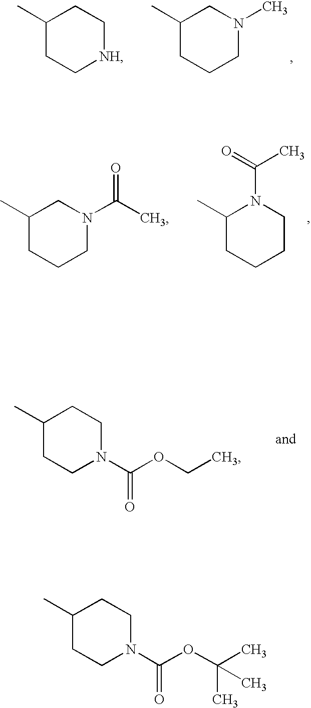
with the proviso R46, R20, and R21 are selected such that the carbon atom to which they are bound does not contain more than one heteroatom (i.e., R46, R20, and R21 are selected such that the carbon atom to which they are bound contains 0 or 1 heteroatom);R⁴⁴ represents 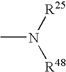
wherein R²⁵ represents heteroaryl, N-methylpiperdinyl or aryl; and R⁴⁸ represents H or alkyl; R⁵⁴ represents an N-oxide heterocyclic group of the formula (i), (ii), (iii) or (iv): 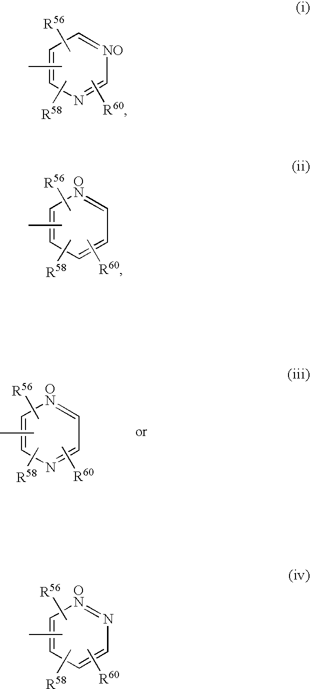
wherein R⁵⁶, R⁵⁸, and R⁶⁰ are the same or different and each is independently selected from H, halo, —CF₃, —OR¹⁰, —C(O)R¹⁰, —SR¹⁰,
—S(O)ₑR¹¹ (wherein e is 1 or 2), —N(R¹⁰)₂, —NO₂, —CO₂R¹⁰, —OCO₂R¹¹, —OCOR¹⁰, alkyl, aryl, alkenyl or alkynyl, which alkyl may be substituted with —OR¹⁰, —SR¹⁰ or —N(R¹⁰)₂ and which alkenyl may be substituted with OR¹¹ or SR¹¹; or R⁵⁴ represents an N-oxide heterocyclic group of the formula (ia), (iia), (iiia) or (iva): 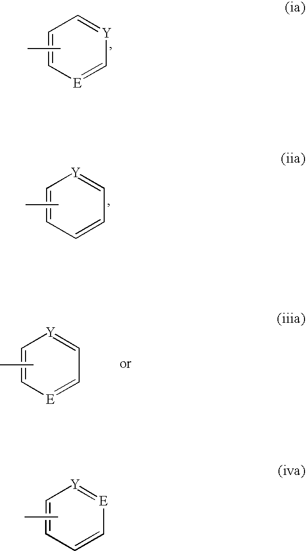
   wherein Y represents N⁺—O⁻ and E represents N; or

R⁵⁴ represents an alkyl group substituted with one of said N-oxide heterocyclic groups (i), (ii), (iii), (iv), (ia), (iia), (iiia) or (iva); Z represents O or S such that R can be taken in combination with R⁵, R⁶, R⁷ or R⁸ as defined above, or R represents R⁴⁰, R⁴², R⁴⁴ or R⁵⁴. Examples of R20, R21, and R46 for the above formulas include:

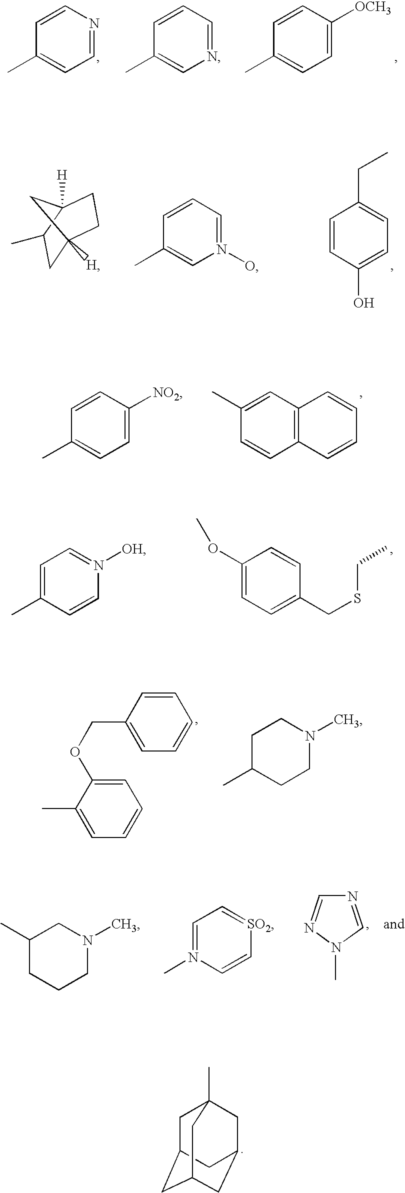

Examples of R25 groups include: 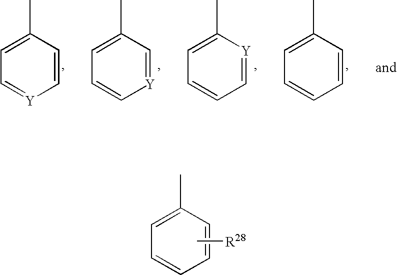
wherein Y represents N or NO, R28 is selected from the group consisting of: C1 to C4 alkyl, halo, hydroxy, NO2, amino (—NH2), —NHR30, and —N(R30)2 wherein R30 represents C1 to C6 alkyl.

In one embodiment, the following tricyclic amide is provided and/or administered in association with the anti-IGF1R formulation of the invention: 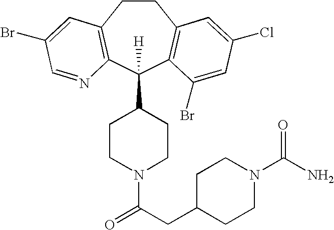
(ionafarnib; Sarasar™; Schering-Plough; Kenilworth, N.J.). In another embodiment, one of the following FPT inhibitors is provided and/or administered in association with the anti-IGF1R formulation of the invention:

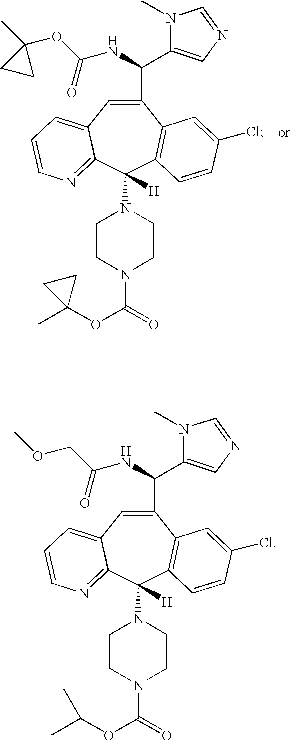

An FPT inhibitor, which, in an embodiment, is provided and/or administered in association with the anti-IGF1R formulation of the invention, includes BMS-214662 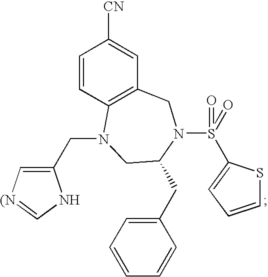
Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

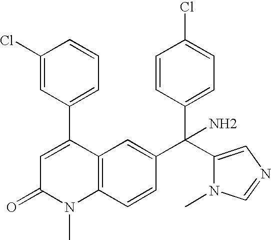

sold as Zarnestra™; Johnson & Johnson; New Brunswick, N.J.).

In an embodiment, an inhibitor which antagonizes the action of the EGF Receptor or HER2, is provided and/or administered in association with the anti-IGF1R formulation of the invention: for example, HuMax-CD20 (sold by Genmab; Copenhagen, Denmark); Campath-1H® (Riechmann et al., Nature 332:323 (1988)); HuMax-EGFr (sold by Genmab; Copenhagen, Denmark); pertuzumab (Omnitarg™, 2C_{4;} Genentech; San Francisco, Calif.); bevacizumab (Presta et al., Cancer Res 57:4593-9 (1997); sold as Avastin®) by Genentech; San Francisco, Calif.); Ibritumomab tiuxetan (sold as Zevalin® by Biogen Idec; Cambridge, Mass.); Tositumomab and Iodine I¹³¹ (sold as Bexxar® by Corixa Corp.; Seattle, Wash. and Glaxosmithkline; Philadelphia, Pa.); gemtuzumab ozogamicin (sold as Mylotarg® by Wyeth Ayerst; Madison, N.J.) or MDX-010 (Medarex; Princeton, N.J.); trastuzumab (sold as Herceptin®; Genentech, Inc.; S. San Francisco, Calif.); CP- 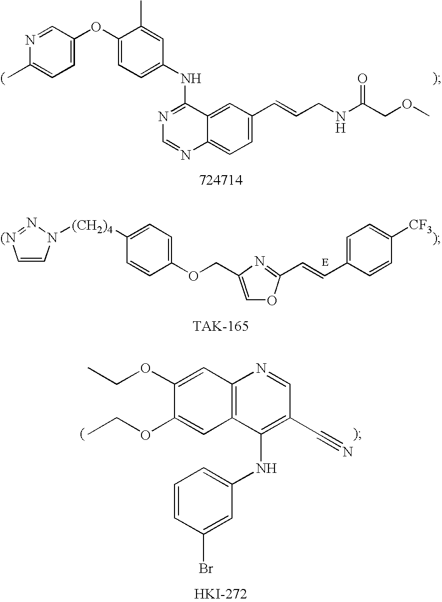
gefitinib (Baselga et al., Drugs 60 Suppl 1:33-40 (2000); ZD-1893; 4-(3-chloro-4-fluoroanilino)-7-methoxy-6-(3-morpholinopropoxy) quinazoline; sold as Iressa™; AstraZeneca; Wilmington, Del.; 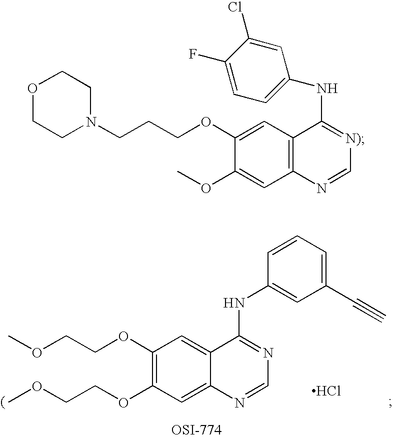
erlotinib, Hidalgo et al., J. Clin. Oncol. 19(13): 3267-3279 (2001)), Lapatanib ( 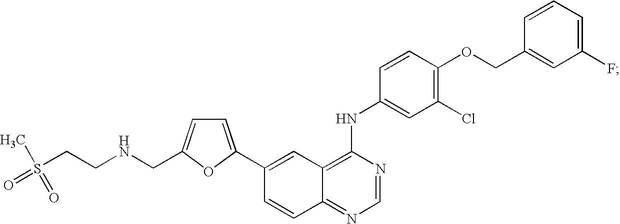
GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (Cl-1033; 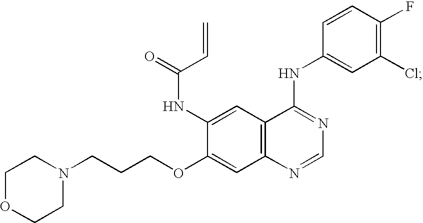
Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):123643 (1999); Yang et al., Crit Rev Oncol Hematol. 38(1):17-23 (2001)), erbitux (U.S. Pat. No. 6,217,866; IMC-C_{225,} cetuximab; Imclone; New York, N.Y.), EKB-569 ( et al., J. Med. Chem. 46(1): 49-63 (2003)), PKI-166 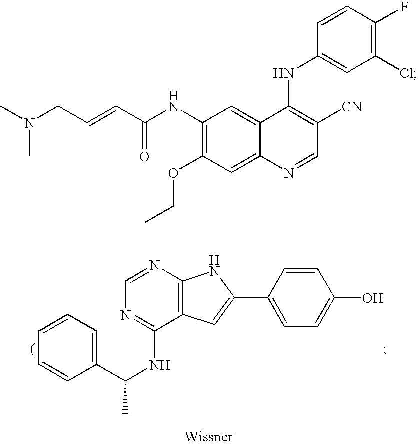
CGP-75166), GW-572016, any anti-EGFR antibody and any anti-HER2 antibody.

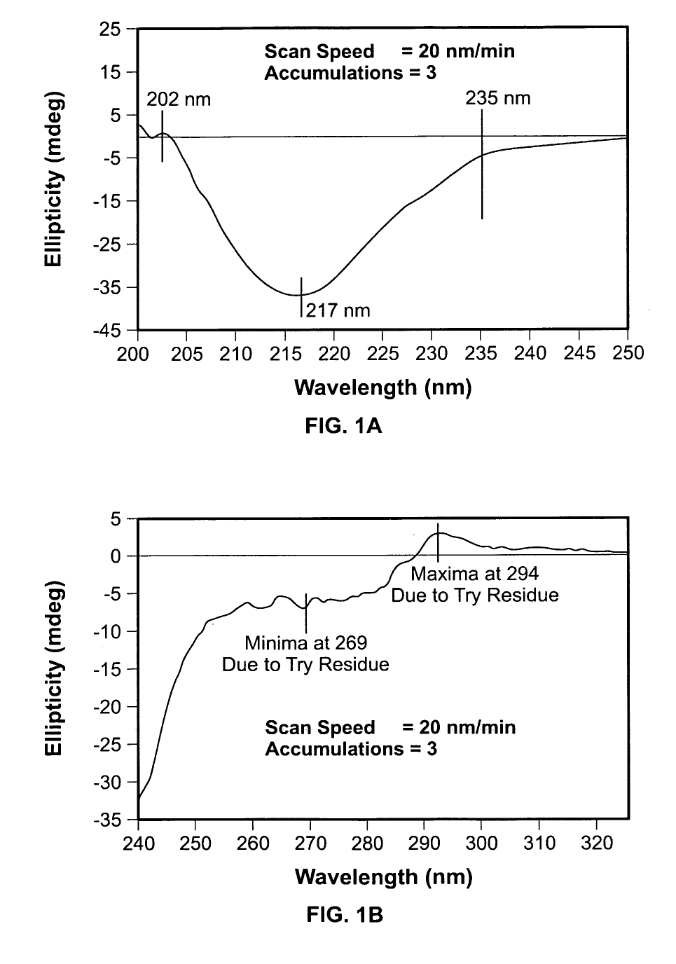

One or more of numerous other small molecules, which have been described as being useful to inhibit EGFR, are, in a embodiment of the invention, provided and/or administered in association with the anti-IGF1R formulation of the invention. For example, U.S. Pat. No. 5,656,655, discloses styryl substituted heteroaryl compounds that inhibit EGFR. U.S. Pat. No. 5,646,153 discloses bis mono and/or bicyclic aryl heteroaryl carbocyclic and heterocarbocyclic compounds that inhibit EGFR and/or PDGFR. U.S. Pat. No. 5,679,683 discloses tricyclic pyrimidine compounds that inhibit the EGFR. U.S. Pat. No. 5,616,582 discloses quinazoline derivatives that have receptor tyrosine kinase inhibitory activity. Fry et al., Science 265 1093-1095 (1994) discloses a compound having a structure that inhibits EGFR (see FIG. 1 of Fry et al.). U.S. Pat. No. 5,196,446, discloses heteroarylethenediyl or heteroarylethenediylaryl compounds that inhibit EGFR. Panek, et al., Journal of Pharmacology and Experimental Therapeutics 283, 1433-1444 (1997) disclose a compound identified as PD166285 that inhibits the EGFR, PDGFR, and FGFR families of receptors. PD166285 is identified as 6- (2,6- dichlorophenyl)-2-(4-(2-diethylaminoethoxy)phenylarnino)-8-methyl-8H- pyrido(2,3-d)pyrimidin-7-one.

In an embodiment of the invention, the anti-IGF1R formulation of the invention is provided and/or administered in association with a LHRH (Lutenizing hormone-releasing hormone) agonist such as the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4]; 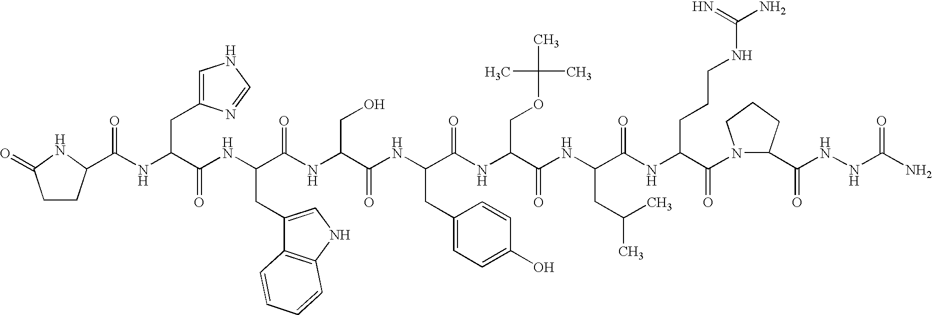
(goserelin acetate; sold as Zoladex® by AstraZeneca UK Limited; Macclesfield, England), 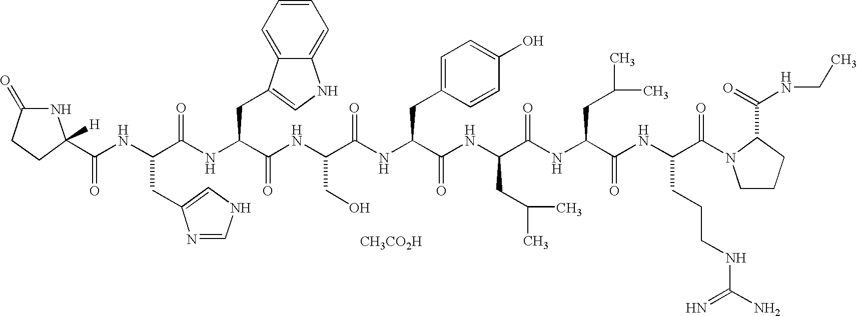
(leuprolide acetate; sold as Eligard® by Sanofi-Synthelabo Inc.; New York, N.Y.) or 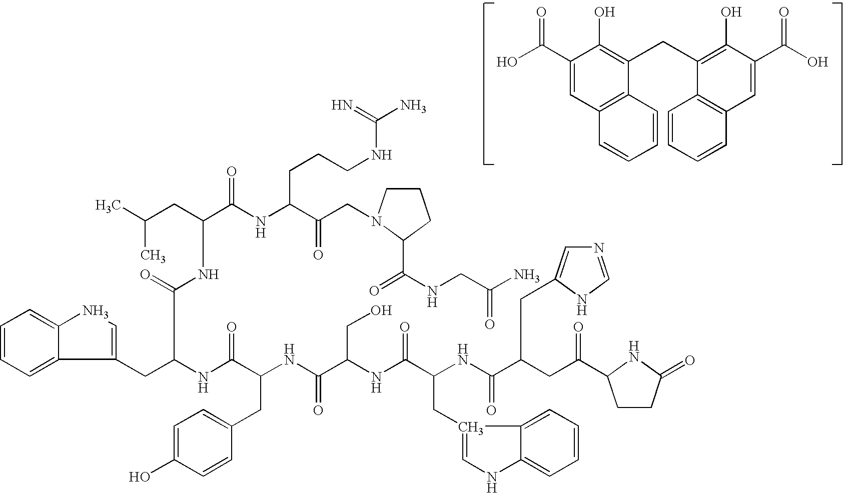
(triptorelin pamoate; sold as Trelistar® by Pharmacia Company, Kalamazoo, Mich.).

In an embodiment of the invention, the anti-IGF1R formulation of the invention is provided and/or administered in association with the FOLFOX regimen (oxaliplatin 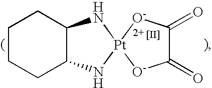
together with infusional fluorouracil 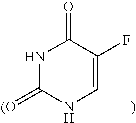
and folinic acid 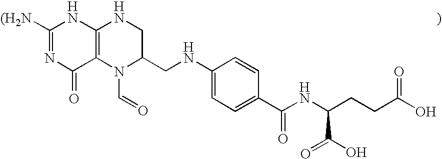
(Chaouche et al., Am. J. Clin. Oncol. 23(3):288-289 (2000); de Gramont et al., J. Clin. Oncol. 18(16):2938-2947 (2000)).

In an embodiment of the invention, the anti-IGF1R formulation of the invention is provided and/or administered in association with 5′-deoxy-5-fluorouridine

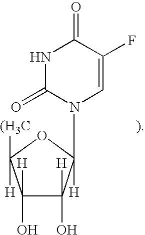

In an embodiment of the invention, the anti-IGF1R formulation of the invention is provided and/or administered in association with Asparaginase; Bacillus Calmette-Guerin (BCG) vaccine (Garrido et al., Cytobios. 90(360):47-65 (1997)); 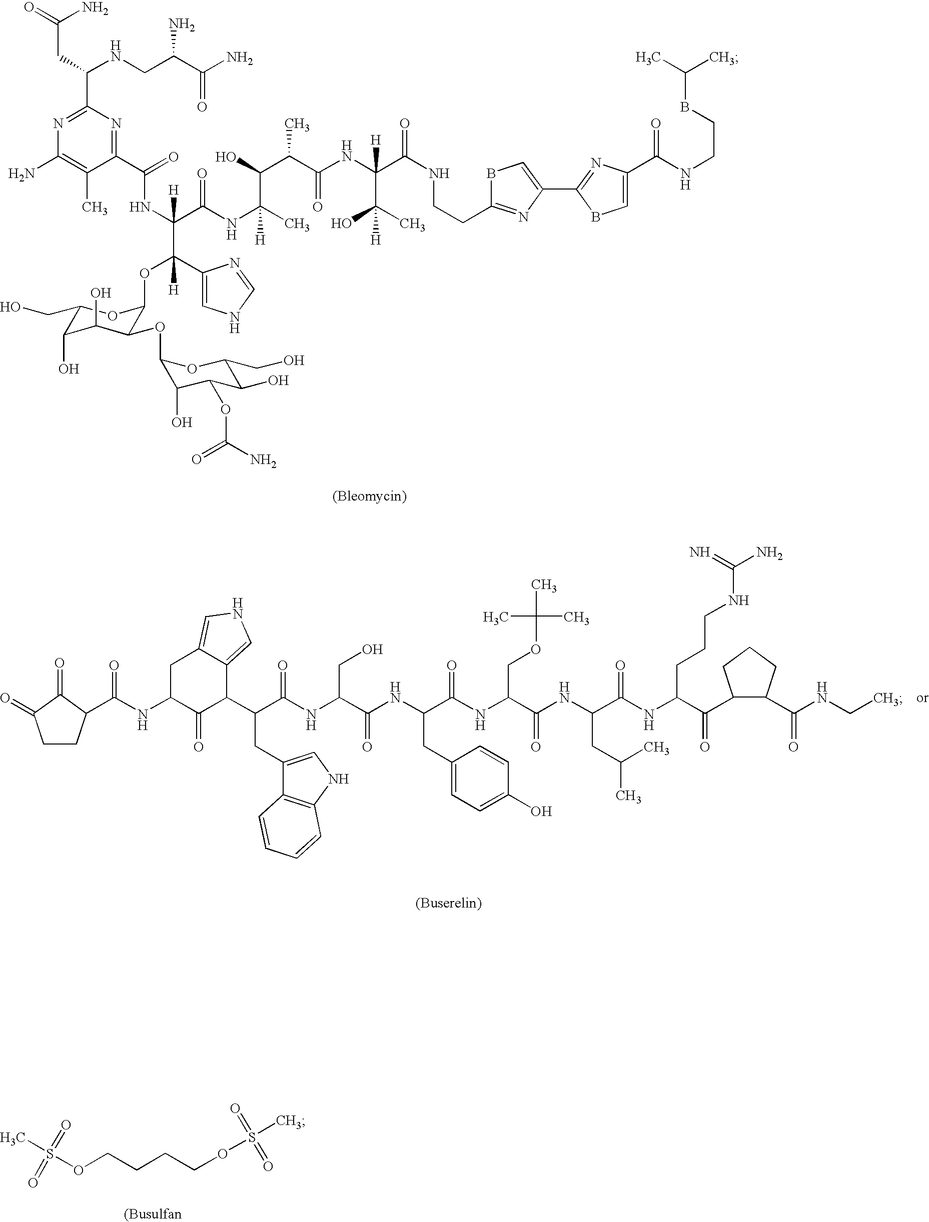
1,4-butanediol, dimethanesulfonate; sold as Busulfex® by ESP Pharma, Inc.; Edison, N.J.).

In an embodiment of the invention, a platinum-based anti-cancer compound, such as oxaliplatin ( 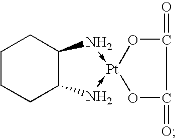
sold as Eloxatin™ by Sanofi-Synthelabo Inc.; New York, N.Y.), 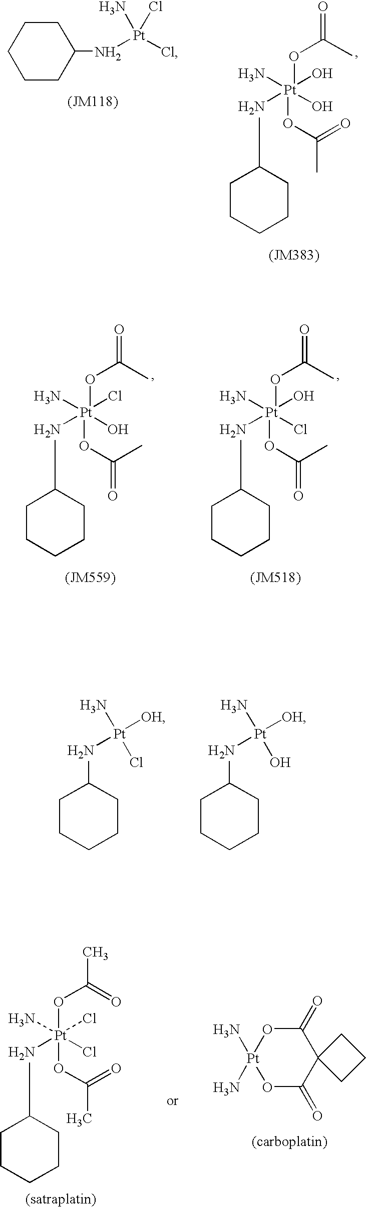
is provided and/or administered in association with the anti-IGF1R formulation of the invention.

In an embodiment of the invention, the anti-IGF1R formulation of the invention is provided and/or administered in association with DES(diethylstilbestrol; 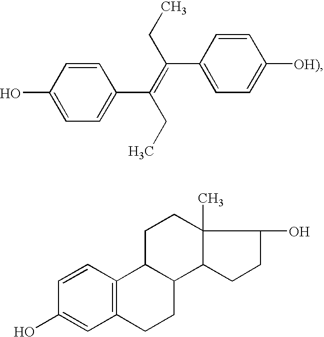
(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, the anti-IGF1R formulation of the invention is provided and/or administered in association with

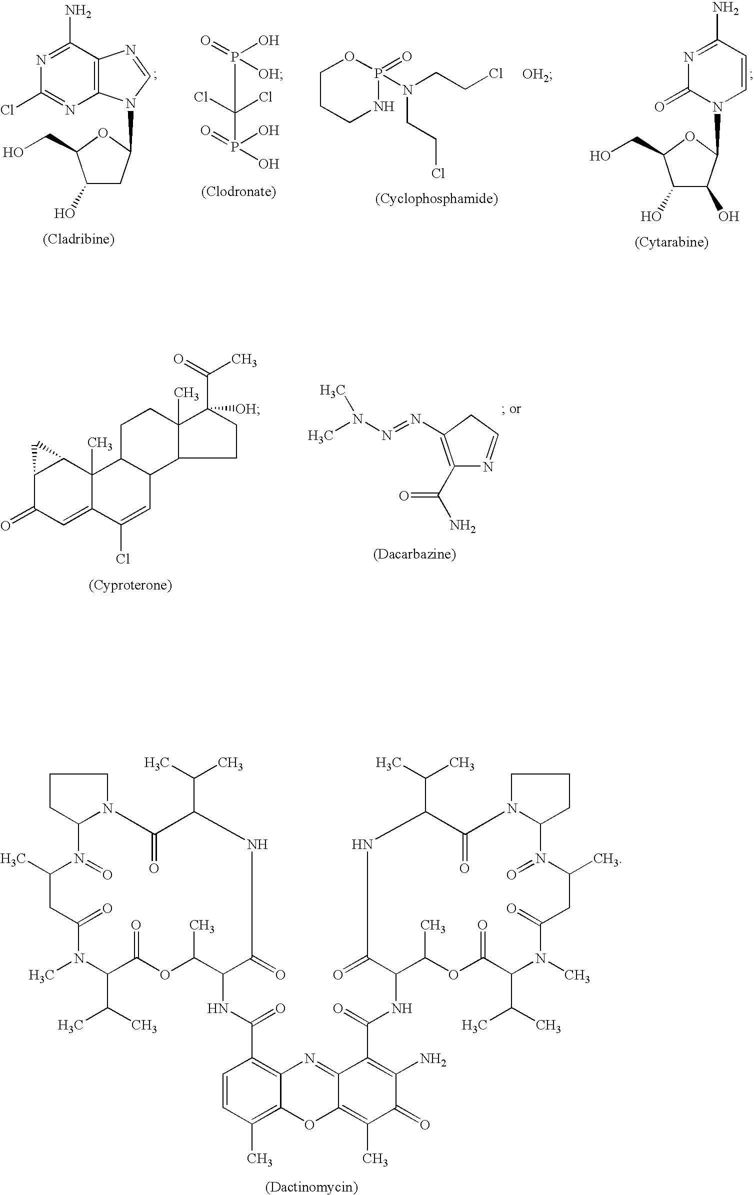

In an embodiment of the invention, a VEGF receptor inhibitor, for example, PTK787/ZK 222584 (Thomas et al., Semin Oncol. 30(3 Suppl 6):32-8 (2003)) or the humanized anti-VEGF antibody Bevacizumab (sold under the brand name Avastin™; Genentech, Inc.; South San Francisco, Calif.) is provided and/or administered in association with the anti-IGF1R formulation of the invention.

In an embodiment of the invention, a MAP kinase inhibitor, for example, VX-745 (Haddad, Curr Opin. Investig. Drugs 2(8):1070-6 (2001)), is provided and/or administered in association with the anti-IGF1R formulation of the invention.

In an embodiment of the invention, a MAP kinase kinase (MEK) inhibitor, such as PD 184352 (Sebolt-Leopold, et al. Nature Med. 5: 810-816 (1999)), is provided and/or administered in association with the anti-IGF1R formulation of the invention.

In an embodiment of the invention, an mTOR inhibitor such as rapamycin or CCI-779 (Sehgal et al., Med. Res. Rev., 14:1-22 (1994); Elit, Curr. Opin. Investig. Drugs 3(8):1249-53 (2002)) is provided and/or administered in association with the anti-IGF1R formulation of the invention.

In an embodiment of the invention, a pl3 kinase inhibitor, such as LY294002, LY292223, LY292696, LY293684, LY293646 (Vlahos et al., J. Biol. Chem. 269(7): 5241-5248 (1994)) or wortmannin is provided and/or administered in association with the anti-IGF1R formulation of the invention.

In an embodiment of the invention, a Raf inhibitor, such as BAY43-9006 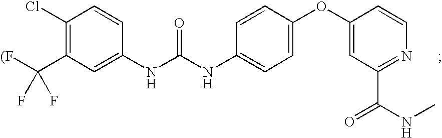
Wilhelm et al., Curr. Pharm. Des. 8:2255-2257 (2002)), ZM336372, L-779,450 or any other Raf inhibitor disclosed in Lowinger et al., Curr. Pharm Des. 8:2269-2278 (2002) is provided and/or administered in association with the anti-IGF1R formulation of the invention.

In an embodiment of the invention, a cyclin dependent kinase inhibitor, such as flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)), is provided and/or administered in association with the anti-IGF1R formulation of the invention.

In an embodiment of the invention, an IGF/IGFR inhibitor, such as an IGF inhibitory peptide (see e.g., U.S. Published Patent Application No. 20030092631 A1; PCT Application Publication NOs. WO 03/27246 A2; WO 02/72780) or any 4-amino-5-phenyl-7-cyclobutyl-pyrrolo[2,3-d] pyrimidine derivative, such as those disclosed in PCT Application Publication No. WO 02/92599 (e.g., 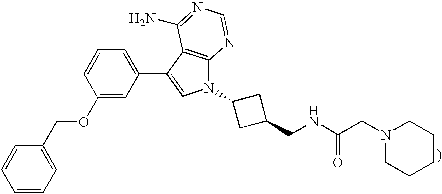
or any flavonoid glycone such as quercetin (see e.g., PCT Application Publication No. WO 03/39538) is provided and/or administered in association with the anti-IGF1R formulation of the invention.

In an embodiment of the invention, the anti-IGF1R formulation of the invention is provided and/or administered in association with 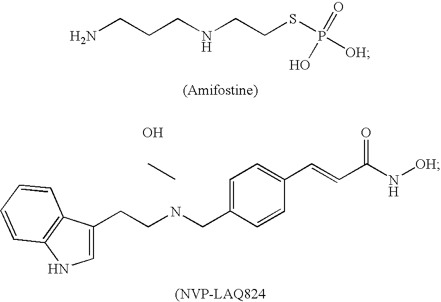
Atadja et al., Cancer Research 64: 689-695 (2004)), 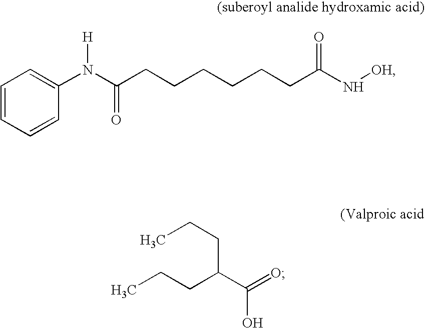
Michaelis et al., Mol. Pharmacol. 65:520-527 (2004)), 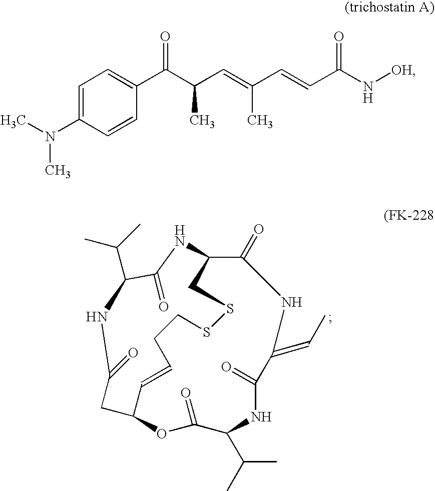
Furumai et al., Cancer Research 62: 4916-4921 (2002)), or 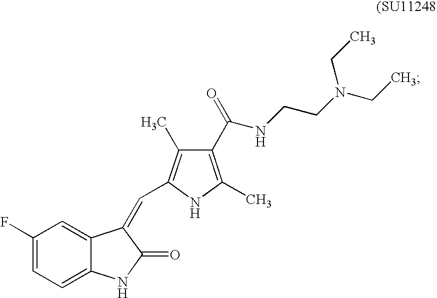
Mendel et al., Clin. Cancer Res. 9(1):327-37 (2003)).

In an embodiment of the invention, the anti-IGF1R formulation of the invention is provided and/or administered in association with a progestational agent such as 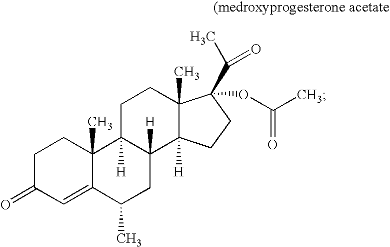
sold as Provera® by Pharmacia & Upjohn Co.; Kalamazoo, Mich.), or 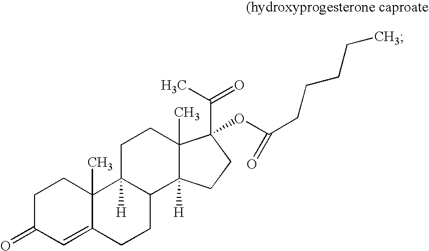
17-((1-Oxohexyl)oxy)pregn-4-ene-3,20-dione;).

In an embodiment of the invention, the anti-IGF1R formulation of the invention is provided and/or administered in association with

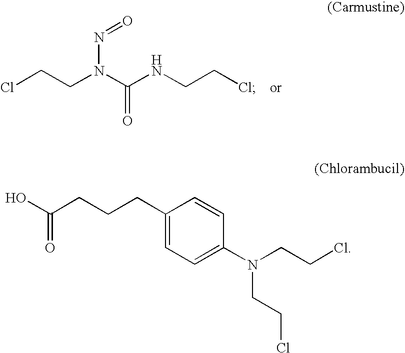

Agents which inhibit IGF production, which, in an embodiment of the invention, are provided and/or administered in association with the anti-IGF1R formulation of the invention, include octreotide (L-Cysteinamide, D-phenylalanyl-L-cysteinyl-L-phenylalanyl-D-tryptophyl-L-lysyl-L-threonyl-N-[2-hydroxy-1-(hydroxymethyl) propyl]-, cyclic (2_{—}7)-disulfide; [R

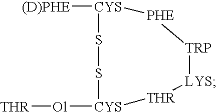

Katz et al., Clin Pharm. 8(4):255-73 (1989); sold as Sandostatin LAR® Depot; Novartis Pharm. Corp; E. Hanover, N.J.).

In an embodiment of the invention, a proteasome inhibitor, such as bortezomib (

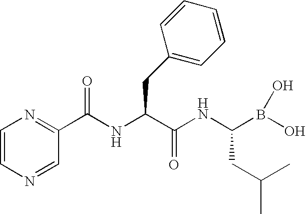

;[(1R)-3-methyl-1-[[(2S)-1-oxo-3-phenyl-2-[(pyrazinylcarbonyl) amino]propyl]amino]butyl] boronic acid; sold as Velcade™; Millennium Pharm., Inc. Cambridge, Mass.), is provided and/or administered in association with the anti-IGF1R formulation of the invention.

In an embodiment of the invention, a microtubule stabilizer or microtubule depolymerizer/inhibitor such as paclitaxel ( 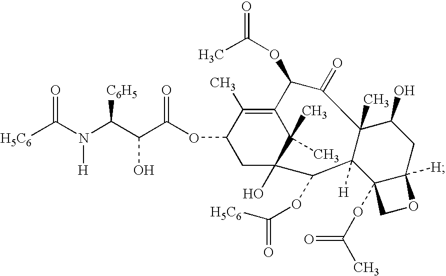
sold as Taxol®; Bristol-Myers Squibb; New York, N.Y.), docetaxel ( 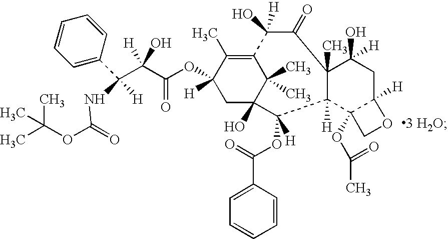
sold as Taxotere®; Aventis Pharm, Inc.; Bridgewater, N.J.); vincristine 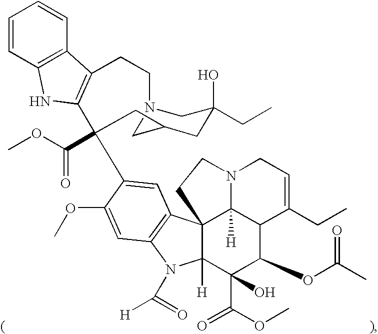
vinblastine 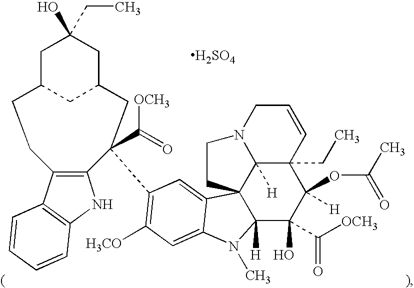
epothilone B and BMS-247550 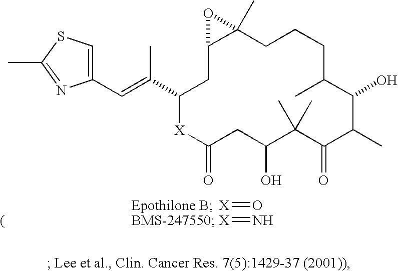
any podophyllotoxin or derivatives thereof including Etoposide (VP-16; 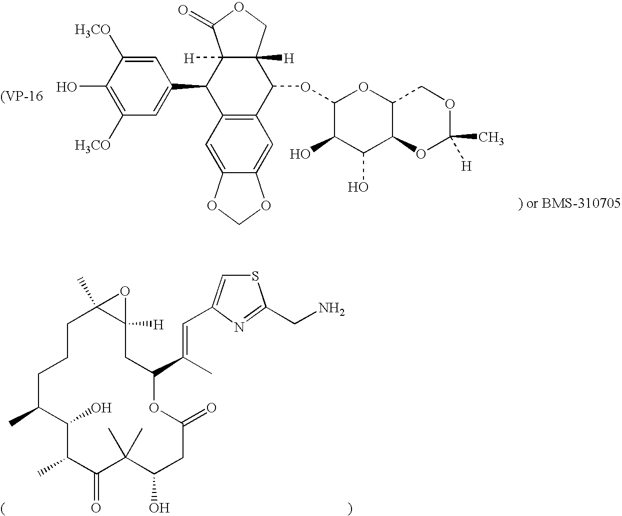
is provided and/or administered in association with the anti-IGF1R formulation of the invention.

In an embodiment of the invention, the anti-IGF1R formulation of the invention is provided in association with any of one or more compounds as set forth in U.S. Pat. No. 5,260,291. For example, in an embodiment of the invention, the compound is a [³H-imidazo-5,1-d]-1,2,3,5-tetrazin-4-one derivative represented by the structural formula:

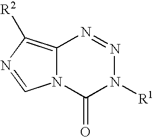

wherein R¹ represents a hydrogen atom, or a straight- or branched-chain alkyl (e.g., —CH₃), alkenyl or alkynyl group containing up to 6 carbon atoms, each such group being unsubstituted or substituted by from one to three substituents selected from halogen (i.e., bromine, iodine or, preferably, chlorine or fluorine) atoms, straight- or branched-chain alkoxy, (e.g., methoxy), alkylthio, alkylsullihinyl and alkylsulphonyl groups containing up to 4 carbon atoms, and optionally substituted phenyl groups, or R¹ represents a cycloalkyl group, and R² represents a carbamoyl group which may carry on the nitrogen atom one or two groups selected from straight- and branched-chain alkyl and alkenyl groups,each containing up to 4 carbon atoms, and cycloalkyl groups, e.g., a methylcarbamoyl or dimethylcarbamoyl group.

When the symbol R¹ represents an alkyl, alkenyl or alkynyl group substituted by two or three halogen atoms, the aforesaid halogen atoms may be the same or different. When the symbol R¹ represents an alkyl, alkenyl or alkynyl group substituted by one, two or three optionally substituted phenyl groups the optional substituents on the phenyl radical(s) may be selected from, for example, alkoxy and alkyl groups containing up to 4 carbon atoms (e.g., methoxy and/or methyl group(s)) and the nitro group; the symbol R¹ may represent, for example, a benzyl or p-methoxybenzyl group. Cycloalkyl groups within the definitions of symbols R¹ and R² contain 3 to 8, preferably 6, carbon atoms.

In an embodiment, tetrazine derivatives of the structural formula 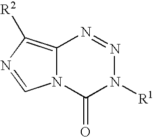
are those wherein R¹ represents a straight-or branched-chain alkyl group containing from 1 to 6 carbon atoms optionally substituted by one or two halogen (preferably chlorine, fluorine or bromine) atoms or by an alkoxy group containing 1 to 4 carbon atoms (preferably methoxy) or by a phenyl group (optionally substituted by one or two alkoxy groups containing from 1 to 4 carbon atoms, preferably methoxy), or R¹ represents an alkenyl group containing 2 to 6 carbon atoms (preferably allyl) or a cyclohexyl group.

In an embodiment, tetrazine derivatives are those of structural formula 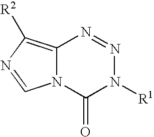
wherein R¹ represents a straight- or branched-chain alkyl group containing from 1 to 6 carbon atoms, and more especially from 1 to 3 carbon atoms, unsubstituted or substituted by a halogen, preferably chlorine or fluorine, atom. In an embodiment, R¹ represents a methyl or 2-haloalkyl, e.g., 2-fluoroethyl or, preferably,2-chloroethyl, group.

In an embodiment, R² represents a carbamoyl group or a monoalkylcarbamoyl, e.g., methylcarbamoyl, or monoalkenylcarbamoyl group.

Temozolomide ( 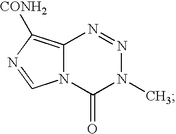
sold by Schering Corp.; Kenilworth, N.J. as Temodar®); 8-carbamoyl-3-methyl-[3H]-imidazo[5,1-d]-1,2,3,5-tetrazin-4-one; 8-carbamoyl-3-n-propyl-[3H]-imidazo[5,1-d]-1,2,3,5-tetrazin-4-one; 8-carbamoyl-3-(2-chloroethyl)-[3H]-imidazo-[5,1-d]-1,2,3,5-tetrazin-4-one; 3-(2-chloroethyl)-8-methylcarbamoyl-[3H]-imidazo[5,1-d]-1,2,3,5-tetrazin-4-one; 8-carbamoyl-3-(3-chloropropyl)-[3H]-imidazo-[5,1-d]-1,2,3,5-tetrazin-4-one; 8-carbamoyl-3-(2,3-dichloropropyl)-[3H]-imidazo[5,1-d]-1,2,3,5-tetrazin-4-one; 3-allyl-8-carbamoyl-[3H]-imidazo[5,1-d]-1,2,3,5-tetrazin-4-one; 3-(2-chloroethyl)-8-dimethylcarbamoyl-[3H]-imidazo[5,1-dl-1,2,3,5-tetrazin-4-one; 3-(2-bromoethyl)-8-carbamoyl-[3H]-imidazo-5,1-d]-1,2,3,5-tetrazin-4-one; 3-benzyl-8-carbamoyl-[3H]-imidazo[5,1-d]-1,2,3,5-tetrazin-4-one; 8-carbamoyl-3-(2-methoxyethyl)-[3H]-imidazo[5,1-d]-1,2,3,5-tetrazin-4-one; 8-carbamoyl-3-cyclohexyl-[3H]-imidazo[5,1-d]-1,2,3,5-tetrazin-4-one; or 8-carbamoyl-3-(Wmethoxybenzyl)-[3H]imidazo[5,1-d]-1,2,3,5-tetrazin-4-one is, in an embodiment of the invention, administered and/or provided with the anti-IGF1R formulation of the invention.

Anthracyclihes which, in an embodiment of the invention, are provided and/or administered in association with the anti-IGF1R formulation of the invention include 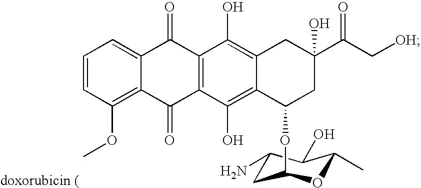
sold as Doxil®; Ortho Biotech Products L.P.; Raritan, N.J.); 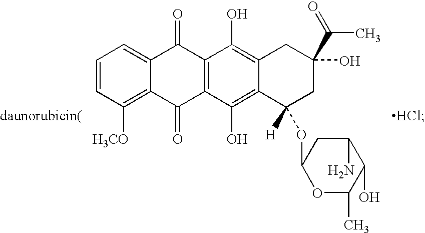
sold as Cerubidine®; Ben Venue Laboratories, Inc.; Bedford, Ohio) and 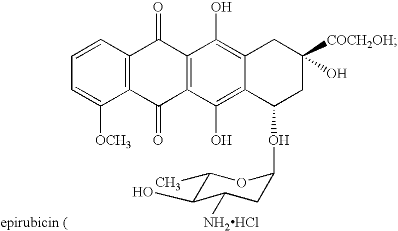
sold as Ellence®; Pharmacia & Upjohn Co; Kalamazoo, Mich.).

In an embodiment of the invention, the anti-IGF1R formulation of the invention is provided and/or administered in association with an anti-androgen including, but not limited to: 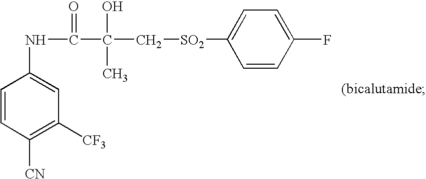
sold at CASODEX® by
AstraZeneca Pharmaceuticals LP; Wilmington, Del.); 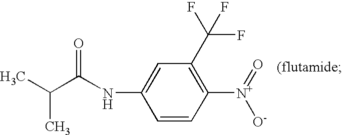
2-methyl-N-[4-nitro-3 (trifluoromethyl)phenyl] propanamide; sold as Eulexin® by Schering Corporation; Kenilworth, N.J.); 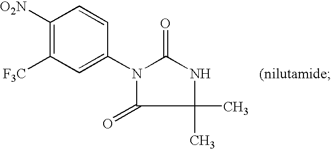
sold as Nilandron® by Aventis Pharmaceuticals Inc.; Kansas City, Mo.) and 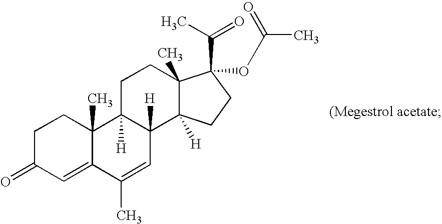
sold as Megace® by Bristol-Myers Squibb).

In an embodiment of the invention, the anti-IGF1R formulation of the invention is provided in association with 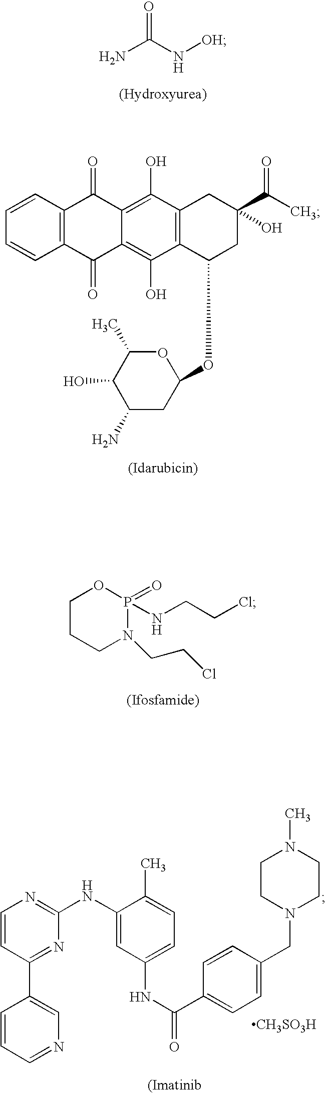
sold as Gleevec® by Novartis Pharmaceuticals Corporation; East Hanover, N.J.); 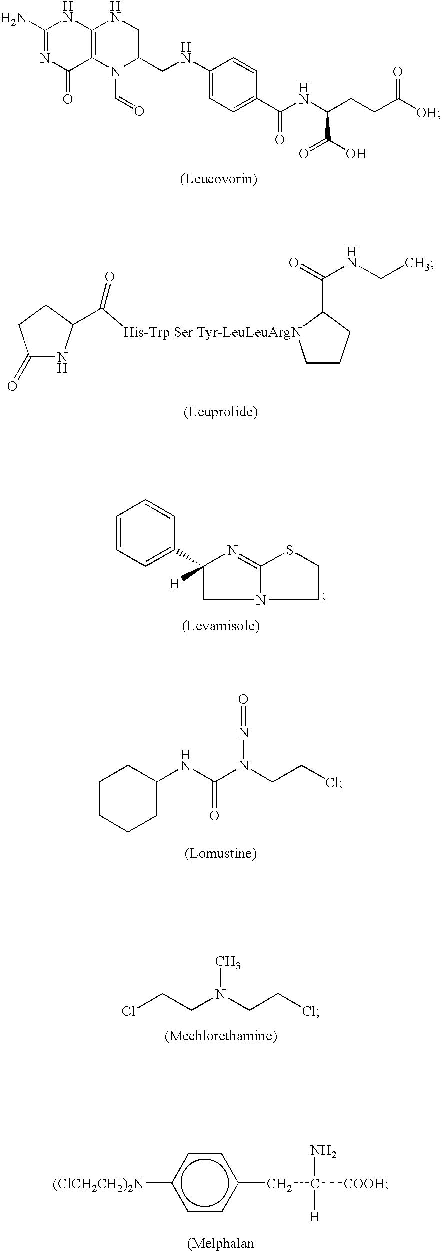
sold as Alkeran® by Celgene Corporation; Warren, N.J.);

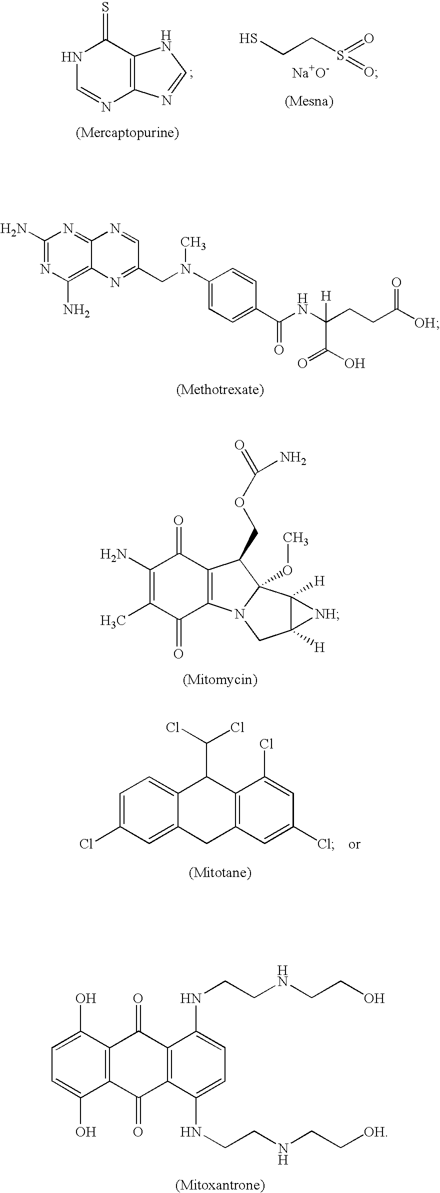

In an embodiment of the invention, the anti-IGF1R formulation of the invention is provided and/or administered in association with

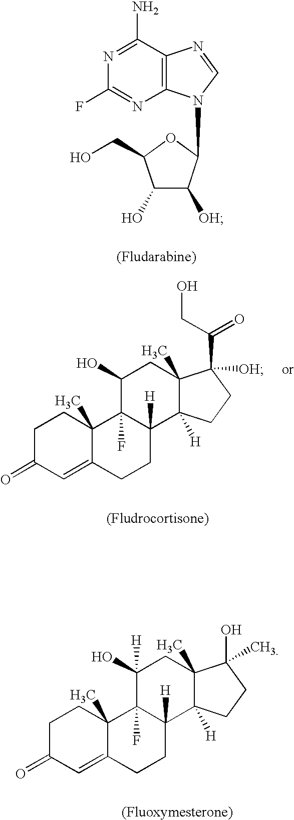

In an embodiment, the anti-IGF1R formulation of the invention is provided and/or administered in association with);;

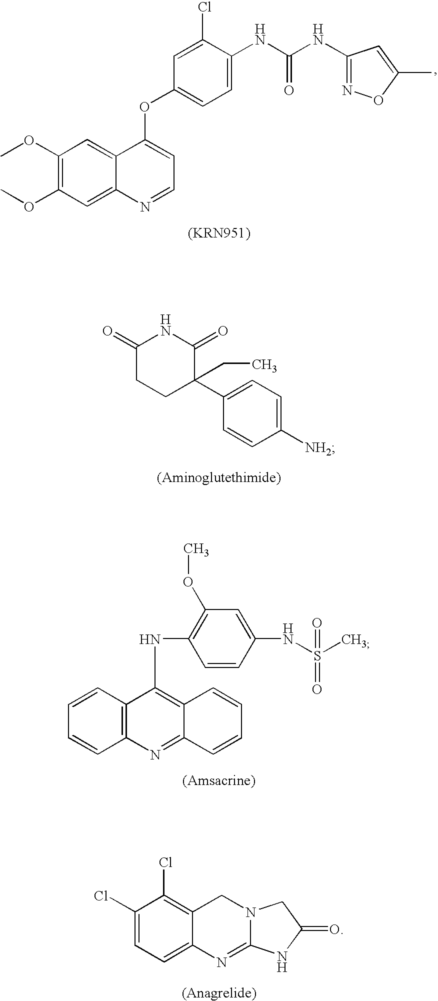

Anti-estrogens and selective estrogen receptor modulators (SERMs), which, in an embodiment of the invention, are administered and/or provided in association with an anti-IGF1R formulation of the invention include droloxifene (3-hydroxytamoxifen), 4- 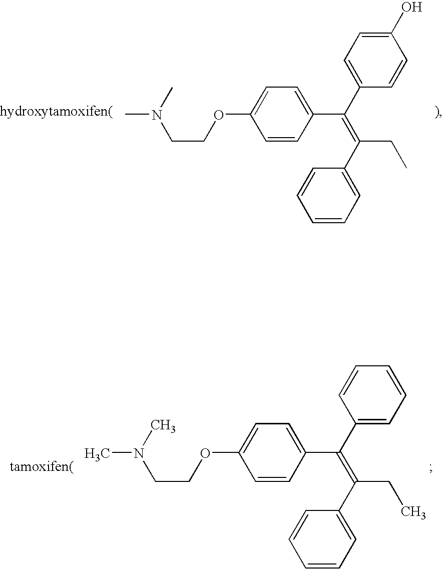
sold as Nolvadex®; Astra Zeneca; Wilmington, Del.); pipendoxifene ( 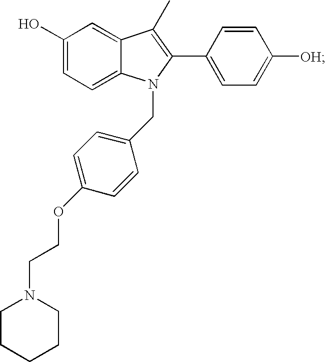
ERA-923; Greenberger et al., Clin. Cancer Res. 7(10):3166-77 (2001)); arzoxifene 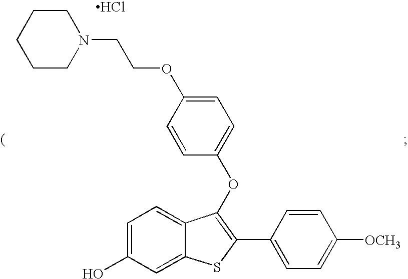
LY353381; Sato et al., J. Pharmacol. Exp. Ther. 287(1):1-7 (1998)); 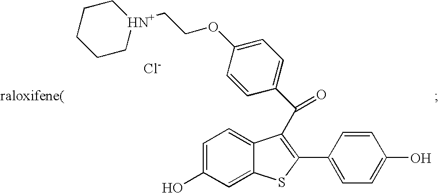
sold as Evista®; Eli Lilly & Co.;
Indianapolis, Ind.); 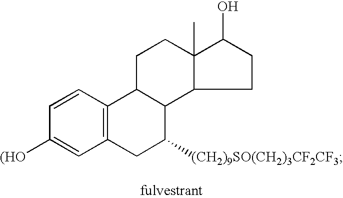
ICI-182780; sold as Faslodex; Astra Zeneca; Wilmington, Del.); acolbifene (EM-652; 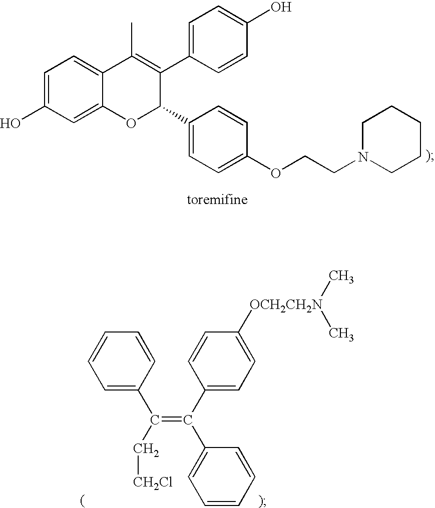
lasofoxifene (CP-336,156; 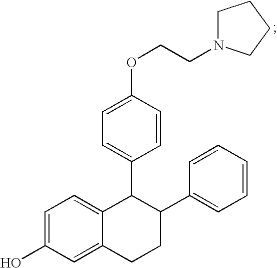
Ke et al., Endocrinology 139(4):2068-76 (1998)); idoxifene (pyrrolidino-4-iodotamoxifen; 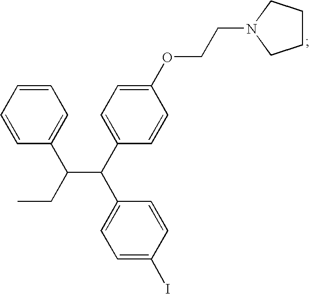
Nuttall et al., Endocrinology 139(12):5224-34 (1998)); TSE-424 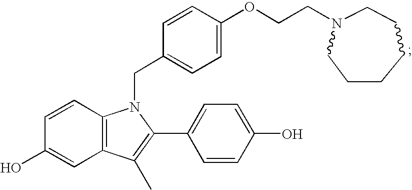
Bazedoxifene; WAY-140424); HMR-3339 and ZK-186619.

Aromatase inhibitors, which can be included with the anti-IGF1R formulation of the invention, include anastrazole 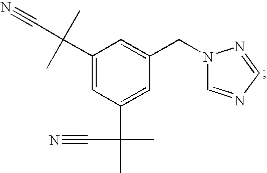
Dukes et al., J. Steroid. Biochem. Mol. Biol. 58(4):439-45 (1996)), letrozole 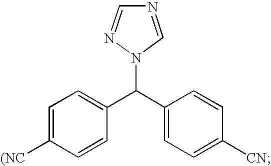
sold as Femara®; Novartis Pharmaceuticals Corp.; E. Hanover, N.J.) and exemestane ( 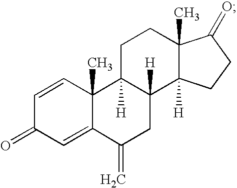
sold as Aromasin®; Pharmacia Corp.; Kalamazoo, Mich.).

The anti-IGF1R formulation of the invention is, in an embodiment of the invention, provided and/or administered in association with gemcitabine HCl 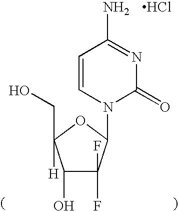
with 13-cis-retinoic acid 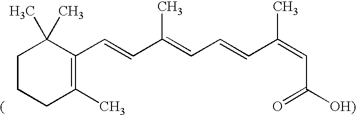
or with any IGFR inhibitor set forth in any of Mitsiades et al., Cancer Cell 5:221-230 (2004); Garcia-Echeverria et. al., Cancer Cell 5:231-239,2004; WO 2004/030627 or WO 2004/030625.

In an embodiment of the invention, the anti-IGF1R formulation of the invention is provided and/or administered in association with 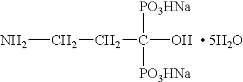
(Pamidronate; sold as Aredia® by Novartis Pharmaceuticals Corporation; East Hanover, N.J.); 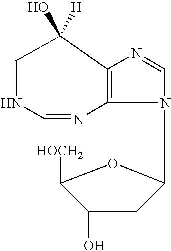
(Pentostatin; sold as Nipent® by Supergen; Dublin, Calif.); 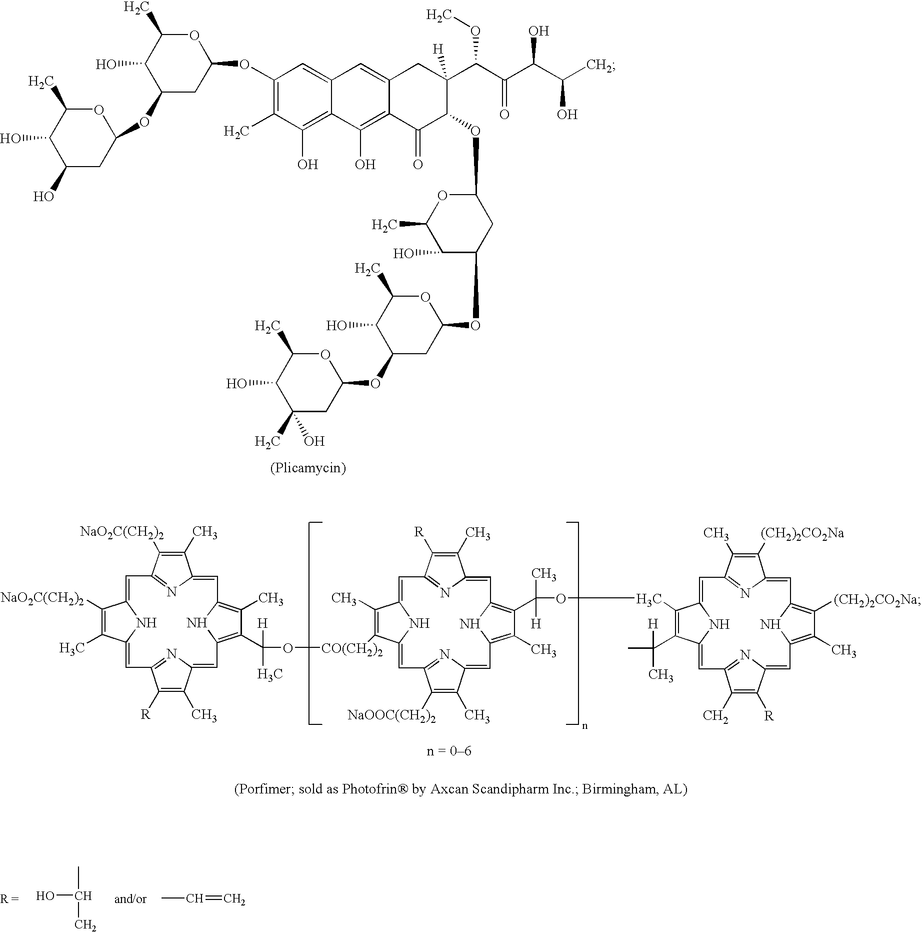
sold as Photofrin® by Axcan Scandipharm Inc.; Birmingham, Ala.); 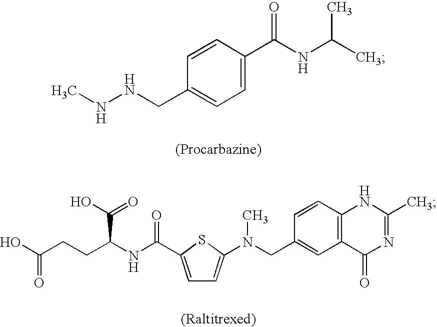
Rituximab (sold as Rituxan® by Genentech, Inc.; South San Francisco, Calif.);

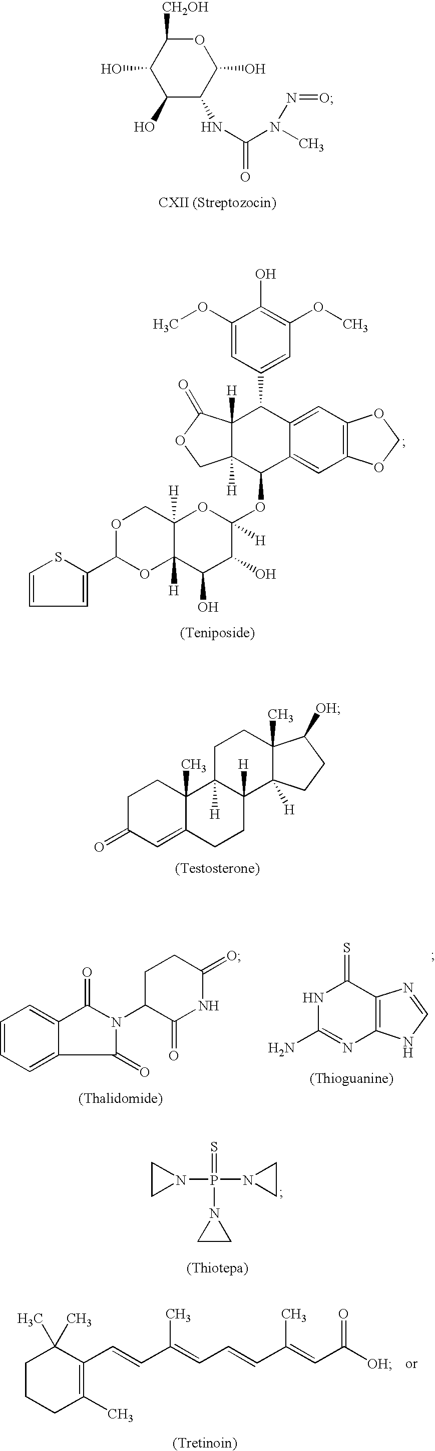

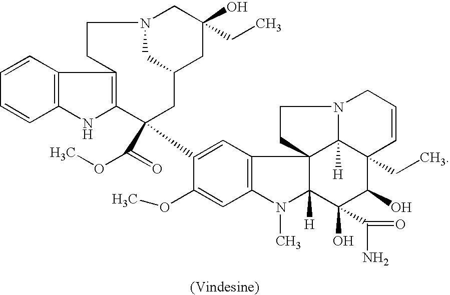

In an embodiment of the invention, the anti-IGF1R formulation of the invention is provided and/or administered in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or album in-interferon-alpha.

Topoisomerase inhibitors which, in an embodiment of the invention, are provided and/or administered in association with an anti-IGF1R formulation of the invention include camptothecin 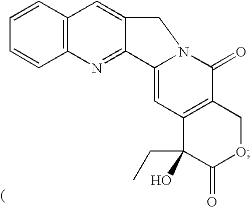
Stork et al., J. Am. Chem. Soc. 93(16): 4074-4075 (1971); Beisleretal., J. Med. Chem. 14(11): 1116-1117 (1962)), topotecan 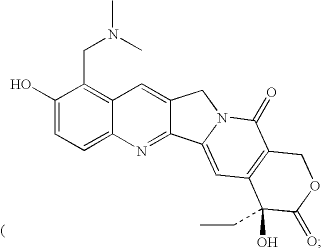
sold as Hycamtin®; GlaxoSmithKline, Research Triangle Park, N.C.; Rowinski et al., J. Clin. Oncol. 10(4): 647-656 (1992)), etoposide 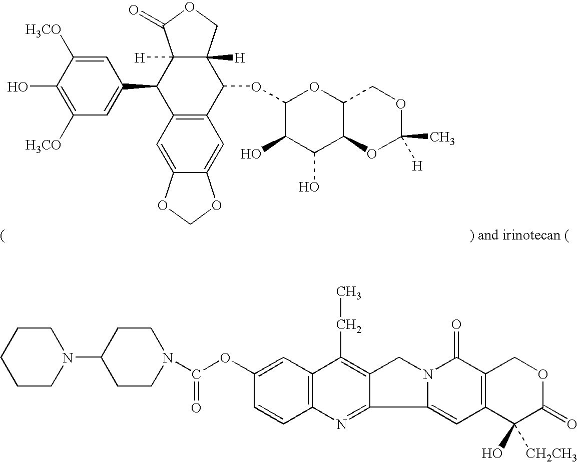
sold as Camptosar®; Pharmacia & Upjohn Co.; Kalamazoo, Mich.).

In an embodiment, an IGF1R1 inhibitory agent provided and/or administered in association with the anti-IGF1R formulation of the invention includes AEW-541 (NVP-AEW-541; NVP-AEW-541-NX-7): 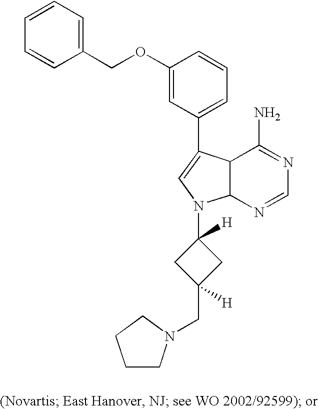
East Hanover, N.J.; see WO 2002/92599); or

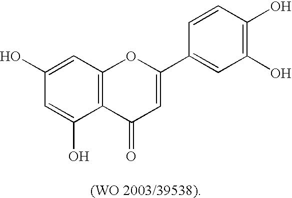

In an embodiment of the invention the anti-IGF1R formulation of the invention is provided and/or administered in association with any kinase inhibitor compound set forth in published international applications WO 2004/030627 or WO 2004/030625. In an embodiment, the kinase inhibitor is (±)-4-[2-(3-chloro-4-fluoro-phenyl)-2-hydroxy-ethylamino]-3-[6-(imidazol-1-yl)-4-methyl-1H-benzimidazol-2-yl]-1H-pyridin-2-one:

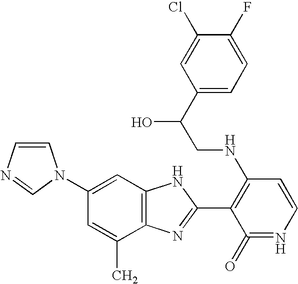

Antisense oligonucleotides can be produced that are complementary to the mRNA of the IGF1R, IGF-1 or IGF-2 gene and can be used to inhibit transcription or translation of the genes. Production of antisense oligonucleotides effective for therapeutic uses is well known in the art. Antisense oligonucleotides are often produced using derivatized or modified nucleotides in order to increase half-life or bioavailability. The primary sequence of the IGF1R, IGF-1 or IGF-2 gene can also be used to design ribozymes. Most synthetic ribozymes are generally hammerhead, tetrahymena and haripin ribozymes. Methods of designing and using ribozymes to cleave specific RNA species are well known in the art. In an embodiment of the invention, the anti-IGF1R formulation of the invention is provided and/or administered in association with the anti-sense IGF1R nucleic acid ATL-1101 (Antisense Therapeutics Ltd; Australia). In an embodiment, the IGF1R anti-sense nucleic acid comprise any of the following nucleotide sequences: 5′-ATCTCTCCGCTTCCTTTC-3′ (SEQ ID NO: 18), 5′-ATCTCTCCGCTTCCTTTC-3′ (SEQ ID NO: 19), 5′-ATCTCTCCGCTTCCTTTC-3′ (SEQ ID NO: 20) or any IGFR antisense nucleic acid set forth in any of US Published Patent Application No. US20030096769; Published International Application No. WO 2003/100059 Fogarty et al., Antisense Nucleic Acid Drug Dev. 2002 December; 12(6):369-77; White et al., J Invest Dermatol. 2002 June; 118(6):1003-7; White et al., Antisense Nucleic Acid Drug Dev. 2000 June; 10(3):195-203; or Wraight et al., Nat Biotechnol. 2000 May; 18(5):521-6.

The chemical structures and other useful information regarding many of the foregoing agents can be found in the Physicians' Desk Reference, 57^{th} ed., 2003; Thompson PDR; Montvale, N.J.

Categorization of a particular agent into a particular class (e.g., FPT inhibitor or microtubule stabilizer) is only done for descriptive purposes and is not meant to limit the invention in any way.

The scope of the present invention also includes compositions comprising the anti-IGF1R formulation of the invention in association with one or more other chemotherapeutic agents (e.g., as described herein) and in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®)), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron ( sold as Zofran® by by Glaxosmithkline; Research Triangle Park, N.C.), dolasetron (sold as Anzemet® by Sanofi-Aventis; New York, N.Y.), or tropisetron (sold as Navoban® by Novartis; East Hanover, N.J.).

The scope of present invention includes compositions and methods comprising the anti-IGF1R formulation of the invention along with one or more of the foregoing chemotherapeutic agents or any salt, hydrate, isomer, formulation, solvate or prodrug thereof.

The scope of the present invention also includes administration of the anti-IGF1R formulation of the invention in association with any anti-cancer procedure including, but not limited to, surgical tumorectomy or anti-cancer radiation therapy.

### Dosage and Administration

Methods of the present invention include provision and/or administration of an IGF1R antibody in a pharmaceutical formulation as set forth herein, optionally in association with a further therapeutic agent, or a pharmaceutical composition thereof to treat or prevent cancer or any medical disorder mediated by IGF1R, IGF-1 and/or IGF-2. Typically, the administration and dosage of such further agents is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

In an embodiment, a formulation of the invention is administered to a subject parenterally, for example, by intravenous, intrathecal, subcutaneous, intramuscular, intratumoral or intraarterial injection. In an embodiment, the formulation is administered orally or by inhalation. In an embodiment of the invention, a formulation of the invention comprising a single-chain anti-IGF1R antibody of the invention is administered pulmonarily by inhalation.

The term “cancer” includes, but is not limited to, neuroblastoma, rhabdomyosarcoma, osteosarcoma, any pediatric cancer, acromegaly, ovarian cancer, pancreatic cancer, benign prostatic hyperplasia, breast cancer, prostate cancer, bone cancer, lung cancer, gastric cancer, colorectal cancer, cervical cancer, synovial sarcoma, bladder cancer, Wilm's cancer, ovarian cancer, benign prostatic hyperplasia (BPH), diarrhea associated with metastatic carcinoid and vasoactive intestinal peptide secreting tumors (e.g., VIPoma or Werner-Morrison syndrome), kidney cancer (e.g., renal cell carcinoma or transitional cell cancer), Ewing Sarcoma, leukemia (e.g., acute lymphoblastic leukemia) or brain cancer (e.g., glioblastoma or a non-glioblastoma) including meningiomas, pituitary adenomas, vestibular schwannomas, primitive neuroectodermal tumors, medulloblastomas, astrocytomas, oligodendrogliomas, ependymomas, and choroid plexus papillomas and any metastatic tumor thereof. Acromegaly may also be treated with a composition of the invention. Antagonism of IGF-I has been reported for treatment of acromegaly (Drake, et al., (2001) Trends Endocrin. Metab. 12: 408413). Other non-malignant medical conditions which may also be treated, in a subject, by administering a formulation of the invention, include gigantism, psoriasis, atherosclerosis, smooth muscle restenosis of blood vessels or inappropriate microvascular proliferation, such as that found as a complication of diabetes, especially of the eye rheumatoid arthritis, Grave's disease, multiple sclerosis, systemic lupus erythematosus, Hashimoto's thyroiditis, myasthenia gravis, auto-immune thyroiditis and Bechet's disease.

The term “therapeutically effective amount” or “therapeutically effective dosage” means that amount or dosage of a composition of the invention (e.g., anti-IGF1R antibody in the formulation of the invention) that will elicit a biological or medical response of a tissue, system, subject or host that is being sought by the administrator (such as a researcher, doctor or veterinarian) which includes any measurable alleviation of the signs, symptoms and/or clinical indicia of a medical disorder, such as cancer (e.g., tumor growth and/or metastasis) including the prevention, slowing or halting of progression of the medical disorder to any degree. For example, in one embodiment, a “therapeutically effective dosage” of any anti-IGF1R antibody (e.g., an anti-IGF1R antibody comprising mature LCC, LCD, LCE or LCF light chain and/or mature HCA or HCB heavy chain) is between about 0.3-20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 3 weeks (e.g., about once every 1 week or once every 2 weeks or once every 3 weeks). As mentioned above, the therapeutically effective dosage of a further therapeutic agent is, when possible, as set forth in the Physicians' Desk Reference.

Dosage regimens may be adjusted to provide the optimum desired response (e.g., a therapeutic response). For example, a single dose may be administered or several divided doses may be administered over time or the dose may be proportionally reduced or increased as indicated by exigencies of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. It is especially advantageous to formulate parenteral compositions in dosage unit form for ease of administration and uniformity of dosage.

A physician or veterinarian having ordinary skill in the art can readily determine and prescribe the effective amount of the pharmaceutical composition required. For example, the physician or veterinarian could start doses of the antibody or antigen-binding fragment of the invention employed in the pharmaceutical composition at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size and progress of a tumor can be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799,1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, neuroblastoma progress can be monitored, by a physician or veterinarian, by a variety of methods, and the dosing regimen can be altered accordingly. Methods by which to monitor neuroblastoma include, for example, CT scan (e.g., to monitor tumor size), MRI scan (e.g., to monitor tumor size), chest X-ray (e.g., to monitor tumor size), bone scan, bone marrow biopsy (e.g.,. to check for metastasis to the bone marrow), hormone tests (levels of hormones like epinephrine), complete blood test (CBC) (e.g., to test for anemia or other abnormality), testing for catecholamines (a neuroblastoma tumor marker) in the urine or blood, a 24 hour urine test for check for homovanillic acid (HMA) or vanillyl mandelic acid (VMA) levels (neuroblastoma markers) and an MIBG scan (scan for injected I¹²³-labeled metaiodobetaguanidine; e.g., to monitor adrenal tumors).

For example, rhabdomyosarcoma progress can be monitored, by the physician or veterinarian, by a variety of methods, and the dosing regimen can be altered accordingly. Methods by which to monitor rhabdomyosarcoma include, for example tumor biopsy, CT scan (e.g., to monitor tumor size), MRI scan (e.g., to monitor tumor size), CT scan of the chest (e.g., to monitor metastases), bone scan (e.g., to monitor metastases), bone marrow biopsy (e.g., to monitor metastases), spinal tap (e.g., to check for metastasis into the brain) and a thorough physical exam.

For example, osteosarcoma progress can be monitored, by the physician or veterinarian, by a variety of methods, and the dosing regimen can be altered accordingly. Methods by which to monitor osteosarcoma include, for example, X-ray of the affected area or of the chest (e.g., to check for spread to the lungs), CT scan of the affected area, blood tests (e.g., to measure alkaline phosphatase levels), CT scan of the chest to see if the cancer has spread to the lungs, open biopsy, or a bone scan to see if the cancer has spread to other bones.

For example, pancreatic cancer progress can be monitored, by the physician or veterinarian, by a variety of methods, and the dosing regimen can be altered accordingly. Methods by which to monitor pancreatic cancer include blood tests to check for tumor markers CA 19-9 and/or carcinoembryonic antigen (CEA), an upper GI series (e.g., a barium swallow), endoscopic ultrasonography; endoscopic retrograde cholangiopancreatography (an x-ray of the pancreatic duct and bile ducts); percutaneous transhepatic cholangiography (an x-ray of the bile duct), abdominal ultrasound imaging or abdominal CT scan.

For example, bladder cancer progress can be monitored, by the physician or veterinarian, by a variety of methods, and the dosing regimen can be altered accordingly. Methods by which to monitor bladder cancer include urinalysis to detect elevated levels of tumor markers (e.g., nuclear matrix protein (NMP22)) in the urine, urinalysis to detect microscopic hematuria, urine cytology to detect cancer cells by examining cells flushed from the bladder during urination, bladder cystoscopy, intravenous pyelogram (IVP), retrograde pyelography, chest X ray to detect metastasis, computed tomography (CT), bone scan, MRI scan, PET scan or biopsy.

For example, breast cancer progress can be monitored, by the physician or veterinarian, by a variety of methods, and the dosing regimen can be altered accordingly. Methods by which to monitor breast cancer include mammography, aspiration or needle biopsy or palpation.

For example, lung cancer progress can be monitored, by the physician or veterinarian, by a variety of methods, and the dosing regimen can be altered accordingly. Methods by which to monitor lung cancer include chest X-ray, CT scan, low-dose helical CT scan (or spiral CT scan), MRI scan, PET scan, bone scan, sputum cytology, bronchoscopy, mediastinoscopy, biopsy (e.g., needle or surgical), thoracentesis or blood tests to detect PTH (parathyroid hormone), CEA (carcinogenic antigen) or CYFRA21-1 (cytokeratin fragment 19).

For example, prostate cancer progress can be monitored, by the physician or veterinarian, by a variety of methods, and the dosing regimen can be altered accordingly. Methods by which to monitor prostate cancer include digital rectal examination, transrectal ultrasound, blood tests taken to check the levels of prostate specific antigen (PSA) and prostatic acid phosphatase (PAP), biopsy, bone scan and CT scan.

For example, colorectal or colon cancer progress can be monitored, by the physician or veterinarian, by a variety of methods, and the dosing regimen can be altered accordingly. Methods by which to monitor colorectal or colon cancer include CT scan, MRI scan, chest X-ray, PET scan, fecal occult blood tests (FOBTs), flexible proctosigmoidoscopy, total colonoscopy, and barium enema.

For example, cervical cancer progress can be monitored, by the physician or veterinarian, by a variety of methods, and the dosing regimen can be altered accordingly. Methods by which to monitor cervical cancer include PAP smear, pelvic exam, colposcopy, cone biopsy, endocervical curettage, X-ray, CT scan, cystoscopy and proctoscopy.

For example, gastric cancer progress can be monitored, by the physician or veterinarian, by a variety of methods, and the dosing regimen can be altered accordingly. Methods by which to monitor gastric cancer include esophagogastroduodenoscopy (EGD), double-contrast barium swallow, endoscopic biopsy, computed tomographic (CT) scanning, magnetic resonance imagine (MRI) or endoscopic ultrasonography (EUS).

For example, Wilm's cancer progress can be monitored, by the physician or veterinarian, by a variety of methods, and the dosing regimen can be altered accordingly. Methods by which to monitor Wilm's cancer include abdominal computer tomography scan (CT), abdominal ultrasound, blood and urine tests to evaluate kidney and liver function, chest X-ray to check for metastasis, magnetic resonance imaging (MRI), blood tests and urinalysis to assay kidney function and biopsy.

In an embodiment of the invention, any patient suffering from a cancer whose tumor cells expresses IGF1R is selected for treatment with a formulation of the invention. In an embodiment of the invention, a patient whose tumor exhibits any of the following characteristics is selected for treatment with a formulation of the invention: IRS-1 phosphorylation on tyrosine 896; (ii) IRS-1 phosphorylation on tyrosine 612; (iii) IRS-1 phosphorylation on any tyrosine; (iv) IGF-II; and/or (v) IGF1R phosphorylation on any tyrosine. Such characteristics can be identified in an tumor cell by any of several methods commonly known in the art (e.g., ELISA or western blot).

### Kits

The kits of the present invention also include an anti-IGF1R antibody formulation of the invention along with information, for example in the form of a package insert, including information concerning the pharmaceutical compositions and dosage forms in the kit. Generally, such information aids patients and physicians in using the enclosed pharmaceutical compositions and dosage forms effectively and safely. For example, the following information regarding formulation can be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information. In an embodiment of the invention, wherein the formulation is provided in dry/lyophilized form, the kit includes sterile water or saline for reconstitution of the formulation into liquid form.

In a kit embodiment of the invention, the anti-IGF1R antibody of the invention is supplied in a vessel (e.g., a vessel that is internally sterile). In an embodiment of the invention, the formulation is in liquid form and in another embodiment of the invention, the formulation of in dry/lyophilized form. The vessel can take any form including, but not limited to, a glass (e.g., sintered glass) or plastic vial or ampule. For example, in an embodiment of the invention the glass is clear and in another embodiment of the invention, the glass is colored (e.g., amber) to block light from contacting the formulation. In an embodiment, the formulation is sparged with nitrogen or an inert gas (e.g., argon). The formulation, in an embodiment, is packaged in a sealed, air-tight vessel under an atmosphere of nitrogen or some inert gas. In an embodiment, the formulation is packaged in an air-tight vessel under vacuum. In an embodiment, the vessel containing the formulation comprises a resealable stopper (e.g., rubber) into which a needle may be inserted for removal of the formulation.

In an embodiment of the invention, the formulation of the invention is provided with an injectable device, for example, a syringe/hypodermic needle. In an embodiment, the syringe is pre-filled with the formulation of the invention (e.g., in liquid or dry/lyophilized form).

In an embodiment, the formulation of the invention is present in a vessel intended for intravenous infusion into the body of a subject. For example, in an embodiment of the invention, the vessel is a plastic infusion bag (e.g., polyvinylchloride or polyethylene).

### EXAMPLES

The following information is provided for more clearly describing the present invention and should not b e construed to limit the present invention. Any and all of the compositions and methods described below fall within the scope of the present invention.

### Example 1

### Formulation and Analysis of Anti-IGF1R Antibody

In this example, an antibody comprising mature light chain LCF (SEQ ID NO: 14 amino acids 20-128), mature heavy chain HCA (SEQ ID NO: 16 amino acids 20-137) and the constant regions (heavy chain γl, light chain κ) was formulated as described and determined to exhibit superior stability characteristics (e.g., exhibiting stability at room temperature for several months).

Method of Manufacture

Materials

1. Sodium Acetate Trihydrate USP: 2.30 g per 1L batch
2. Glacial Acetic Acid USP/Ph. Eur: 0.18 g per 1L batch
3. Sucrose Extra Pure NF, Ph. Eur, BP: 70.0 g per 1L batch
4. Antibody: 20.0 g per 1L batch
5. Water for injection USP/Ph. Eur: quantity sufficient for 1L volume
Note: the anti-IGF1R antibody may be susceptible to aggregation due to foaming and shaking. Avoid excess foaming during manufacturing, filtration and filling.
   Methods
   Compounding

Charged and dissolved sodium acetate trihydrate, acetic acid and sucrose in approximately 70% of batch volume of water for injection at room temperature in a stainless steel tank equipped with an agitator. To this solution, charged the required amount of drug substance (antibody) to the stainless steel vessel and agitated for at least 20 minutes. After agitating for 20 minutes, brough the batch to final volume with water for injection and allowed to agitate for another 20 minutes. Checked the pH of the solution. Aseptically filtered the solution through a sterilized filter (0.22 μm) into a sterilized stainless steel container. Aseptically filled into vials that had been washed and sterilized. Stoppered and crimped the vials with aluminum seals.

Stability Testing

Two batches were manufactured according to the process described in the Compounding section.

The sealed vials from a prototype batch (Batch A) were placed on stability stations at the following conditions: 4 (4±2° C.; 60% ±5% RH), 25H (25±2° C.; 60% ±5% RH and 40 (40±2° C., ambient RH) for 3 months. Initial samples and samples pulled at the end of each time-point were stored at 4° C. prior to analyses.

The sealed vials from a second batch (Batch B) were placed on the same stability stations as Batch A, in both the upright and inverted positions, for 6 months. Initial samples and samples pulled at the end of each time-point were stored at 4° C. prior to analyses.

TABLE 1A
Summary of assay results for anti-IGF1R antibody stability, Batch A.
20 mM Ace pH 5.5 + 7%
Prototype Sucrose

| Sample ID | Initial | 2 wk 40 C. | 4 wk 4 C. | 4 wk 25 C. | 4 wk 40 C. | 12 wk 4 C. | 12 wk 25 C. |
|---|---|---|---|---|---|---|---|
| PhysObs | clear solution | clear | clear | clear | clear | opalescent | opalescent |
| | contains | solution | solution | solution | solution | solution | solution |
| | particles | contains | contains | contains | contains | contains | contains |
| | | particles | particles | particles | particles | particles | particles |
| pH | 5.4 | 5.4 | 5.4 | 5.3 | 5.4 | 5.4 | 5.4 |
| UV (mg/mL) | 22.34 | 22.75 | 24.78 | 23.40 | 22.43 | 22.49 | 23.06 |
| HPSEC | | | | | | | |
| % Monomer | 99.394 | 98.931 | 99.416 | 99.261 | 98.477 | 99.421 | 99.035 |
| % Early Eluting | 0.205 | 0.249 | 0.181 | 0.221 | 0.313 | 0.135 | 0.174 |
| % Late Eluting | 0.402 | 0.82 | 0.404 | 0.518 | 1.211 | 0.445 | 0.792 |
| SDS-PAGE | | | | | | | |
| Reducing | Heavy and | Heavy and | Heavy and | Heavy and | Heavy and | Heavy and | Heavy and |
| | light chains | light | light | light | light | light chains | light chains |
| | detected | chains | chains | chains | chain | detected | detected |
| | under | detected | detected | detected | detected | under | under |
| | reducing | under | under | under | under | reducing | reducing |
| | conditions | reducing | reducing | reducing | reducing | conditions | conditions |
| | | conditions | conditions | conditions | conditions | | |
| Non Reducing | Band pattern | Band | Band | Band | Band | Band | Band |
| | matches | pattern | pattern | pattern | pattern | pattern | pattern |
| | typical non- | matches | matches | matches | matches | matches | matches |
| | reducing | typical | typical | typical | typical | typical non- | typical non- |
| | antibody | non- | non- | non- | non- | reducing | reducing |
| | profile | reducing | reducing | reducing | reducing | antibody | antibody |
| | | antibody | antibody | antibody | antibody | profile | profile |
| | | profile | profile | profile | profile | | |
| Bio Assay (mg/mL) | 21.4 | 18.3 | 14.0 | 17.2 | 11.8 | 23.3 | 29.2 |
| HIAC | | | | | | | |
| Particle Size (≧10 μm) | 387 | | | | | 323 | 437 |
| (Particle | | | | | | | |
| count/container) | | | | | | | |
| Particle Size (≧25 μm) | 27 | | | | | 30 | 35 |
| (Particle | | | | | | | |
| count/container) | | | | | | | |
| Nanosizer | | | | | | | |
| Particle Size (nm) | 12.22 | 14.92 | 14.92 | 14.92 | 12.22 | 11.05 | 11.05 |

TABLE 1B
Summary of assay results for anti-IGF1R antibody stability, Batch B.

| | | 1 Month 5 C. | 1 Month 25 C. | 1 Month 40 C. |
|---|---|---|---|---|
| Sample ID | Initial | (Upright) | (Upright) | (Upright) |
| Description | clear solution | opalescent solution | opalescent solution | opalescent solution |
| | contains particles | contains particles | contains particles | contains particles |
| pH | 5.5 | 5.5 | 5.6 | 5.6 |
| UV (mg/mL) | 19.72 | 18.51 | 18.87 | 18.71 |
| Purity HPSEC | | | | |
| % Monomer | 99.281 | 99.28 | 99.219 | 98.757 |
| % Early Eluting | 0.301 | 0.296 | 0.305 | 0.395 |
| % Late Eluting | 0.419 | 0.425 | 0.476 | 0.849 |
| Purity SDS-PAGE | | | | |
| Reducing (Total Impurity) | 2.73 | 1.05 | 1.28 | 2.15 |
| Non Reducing (Total Impurity) | 17.45 | 12.3 | 15.09 | 14.7 |
| Bio Assay (SPU/mL) | 10.3 mg/mL | 16.46 mg/mL | 20.01 mg/mL | 13.91 mg/mL |
| HIAC | | | | |
| Particle Size (≧10 μm) | 468 | 1161 | 927 | 1069 |
| (Particle count/container) | | | | |
| Particle Size (≧25 μm) | 30 | 87 | 42 | 71 |
| (Particle count/container) | | | | |
| Isoelecdtic Focusing | Band pattern matches | Band pattern | Band pattern | Band pattern |
| (IEF) | the profile of | matches the | matches the | matches the |
| | research batches | profile of | profile of | profile of |
| | | research batches | research batches | research batches |

| | 3 Month 5 C. | 3 Month 25 C. | 3 Month 40 C. | 3 Month 5 C. | 3 Month 25 C. | 3 Month 40 C. |
|---|---|---|---|---|---|---|
| Sample ID | (Upright) | (Upright) | (Upright) | (Inverted) | (Inverted) | (Inverted) |
| Description | opalescent | opalescent | opalescent | opalescent | opalescent | opalescent |
| | solution | solution | solution | solution | solution | solution |
| | contains | contains | contains | contains | contains | contains |
| | particles | particles | particles | particles | particles | particles |
| pH | 5.3 | 5.3 | 5.4 | 5.3 | 5.3 | 5.4 |
| UV (mg/mL) | 18.44 | 18.14 | 17.96 | 18.03 | 18.6 | 18.1 |
| Purity HPSEC | | | | | | |
| % Monomer | 99.266 | 99.07 | 97.593 | 99.288 | 98.049 | 97.613 |
| % Early Eluting | 0.301 | 0.335 | 0.691 | 0.3 | 0.339 | 0.7 |
| % Late Eluting | 0.434 | 0.596 | 1.717 | 0.413 | 0.615 | 1.688 |
| Purity SDS-PAGE | | | | | | |
| Reducing (Total | 1.48 | 2.73 | 7.32 | 1.12 | 1.77 | 7.54 |
| Impurity) | | | | | | |
| Non Reducing (Total | 21.17 | 28.13 | 26.67 | 22.64 | 26 | 29.13 |
| Impurity) | | | | | | |
| Bio Assay (SPU/mL) | 12.93 mg/ml | 15.78 mg/ml | 9.41 mg/ml | 14.13 mg/ml | 13.28 mg/mL | 11.41 mg/ml |
| HIAC | | | | | | |
| Particle Size (≧10 μm) | 965 | 532 | 1800 | 586 | 3836 | 322 |
| (Particle | | | | | | |
| count/container) | | | | | | |
| Particle Size (≧25 μm) | 22 | 18 | 185 | 41 | 175 | 10 |
| (Particle | | | | | | |
| count/container) | | | | | | |
| Isoelecdtic Focusing | Four to five | Four to five | Four to five | Four to five | Four to five | Four to five |
| (IEF) | bands | bands | bands between | bands between | bands between | bands between |
| | between pl | between pl | pl markers 8.3 | pl markers 8.3 | pl markers 8.3 | pl markers 8.3 |
| | markers 8.3 | markers 8.3 | and 9.5 | and 9.5 | and 9.5 | and 9.5 |
| | and 9.5 | and 9.5 | | | | |
| | 6 Month 5 C. | 6 Month 25 C. | 6 Month 40 C. | 6 Month 5 C. | 6 Month 25 C. | 6 Month 40 C. |
| Sample ID | (Upright) | (Upright) | (Upright) | (Inverted) | (Inverted) | (Inverted) |
| Description | opalescent | opalescent | opalescent | opalescent | opalescent | opalescent |
| | solution | solution | solution | solution | solution | solution |
| | contains | contains | contains | contains | contains | contains |
| | particles | particles | particles | particles | particles | particles |
| pH | 5.5 | 5.5 | 5.5 | 5.4 | 5.5 | 5.5 |
| UV (mg/mL) | 19.52 | 16.32 | 19.28 | 18.32 | 18.6 | 16.86 |
| Purity HPSEC | | | | | | |
| % Monomer | 99.235 | 98.851 | 95.62 | 99.3 | 98.837 | 95.723 |
| % Early Eluting | 0.25 | 0.317 | 1.406 | 0.229 | 0.319 | 1.348 |
| % Late Eluting | 0.516 | 0.832 | 2.975 | 0.472 | 0.845 | 2.936 |
| Purity SDS-PAGE | | | | | | |
| Reducing (Total | 1.43 | 3.52 | 12.5 | 1.74 | 3.61 | 12.64 |
| Impurity) | | | | | | |
| Non Reducing (Total | 13.67 | 16.55 | 24.86 | 12.68 | 15.64 | 24.33 |
| Impurity) | | | | | | |
| Bio Assay (SPU/mL) | NA | NA | NA | NA | NA | NA |
| HIAC | | | | | | |
| Particle Size (≧10 μm) | 678 | 424 | 1870 | 1894 | 96 | 1270 |
| (Particle | | | | | | |
| count/container) | | | | | | |
| Particle Size (≧25 μm) | 45 | 35 | 90 | 178 | 2 | 78 |
| (Particle | | | | | | |
| count/container) | | | | | | |
| Isoelectric Focusing | Four to five | Four to five | Four to five | Four to five | Four to five | Four to five |
| (IEF) | bands | bands | bands between | bands between | bands between | bands between |
| | between pl | between pl | pl markers 8.3 | pl markers 8.3 | pl markers 8.3 | pl markers 8.3 |
| | markers 8.3 | markers 8.3 | and 9.5 | and 9.5 | and 9.5 | and 9.5 |
| | and 9.5 | and 9.5 | | | | |

Data Analysis and Reporting
Batch A
Description:

The description ranged from clear solution contains particles up to 4 week samples to opalescent solution contains particles for 3 week samples.

pH:

The pH ranged between 5.3 and 5.4.

UV Conc:

The initial UV concentration obtained was 22.34 mg/mL. The concentration determined by UV assay for the other time points remained constant within 90-110% of the initial value. The differences observed are within the normal variability of this assay.

HPSEC:

The purity assessed by HPSEC assay suggested that for prototype formulation, the percentage monomer content was more than 99% at 4° C. and 25° C. up to 12 weeks. At 40° C., the percentage monomer content decreased to 98.93 and 98.47 after 2 weeks and 4 weeks respectively.

SDS-PAGE:

SDS PAGE results suggested typical band pattern which matches with typical non-reducing antibody profile under non-reducing condition and detection of heavy and light chain was reported under reducing condition for all the time points.

Bioasaay:

Bioassay showed significant variability between results of 4 weeks and 12 weeks. The concentration obtained with this assay reduced to 14.0 mg/mL after 2 weeks at 4° C. as compared to initial concentration of 21.4 mg/mL. On the other hand, after 12 weeks at 4° C., the concentration obtained for prototype formulation 1 was 23.3 mg/mL. The differences observed are within the normal variability of this assay.

HIAC:

The Particulate data met USP <788> specification (Light obscuration test particle count: ≧10 μm—6000 per container, ≧25 μm—600 per container) for all samples.

Particle Sizing:

The particle size of the samples ranged from 11.05 nm to 14.92 nm for all the samples. The differences observed for particle size are within the normal variability of this assay.

Batch B

Description:

The description ranged from clear solution contains particles at initial to opalescent solution contains particles for subsequent samples.

pH:

The pH ranged between 5.3 and 5.5.

UV Conc:

The initial UV concentration obtained was 19.72 mg/mL. The concentration determined by UV assay for the other time points remained within 90-110% of the initial value. The differences observed are within the normal variability of this assay.

HPSEC:

The purity assessed by HPSEC assay suggested that for prototype formulation, the percentage monomer content was more than 98% at 4° C. and 25° C. up to 6 months. At 40° C., the percentage monomer content decreased to about 95% after 6 months.

SDS-PAGE:

Quantitative SDS PAGE results for both reducing and non-reducing conditions show levels of total impurities which remain relatively constant (within the variability of the assay) at 4° C. and 25° C. up to 6 months, with an increase in levels at 40° C. over 6 months.

Bioasaay:

Bioassay showed significant variability over 3 months, with no apparent trend with temperature or time. The differences observed are within the normal variability of this assay.

HIAC:

The Particulate data met USP <788> specification (Light obscuration test particle count: ≧210 μm—6000 per container, ≧25 μm—600 per container) for all samples.

Isoelectric Focusing (IEF):

Isoelectric Focusing measures the charge variations in the antibody molecules. The description of the banding pattern reported at Initial and 1 month is equivalent to the description reported at 3 and 6 months, so the results remain constant over 6 months at all temperatures.

### Example 2

### Stability Study of Anti-IGF1R Formulations

The anti-IGF1R antibody used in these studies was the same as that used in Example 1. Based on these studies, the following was determined:
The anti-IGF1R antibody exhibited predominantly β-sheet secondary structure in all the buffers tested.
The anti-IGF1R antibody showed a high Tₒₙₛₑₜ temperature in a pH range of 5 and 6.
The anti-IGF1R antibody, in acetate buffer with pH 5.5, showed highest onset temperatures.
Addition of sodium chloride decreased onset of thermal denaturation for all the buffers tested.
Addition of sucrose increased onset of thermal denaturation for all the buffers tested.
The anti-IGF1R antibody, in a formula of 20 mM acetate buffer pH 5.5 with 7% w/v sucrose, was stable at 4° C. and 25° C. for 28 days.
   Materials.

A stock solution of the anti-IGF1R antibody (28.36 mg/ml) in 5 mM acetate buffer pH5.2 was used to prepare dilutions in various buffers of pH 4 to 9.

TABLE 2
Summary of buffers and pH conditions under which the anti-IGF1R
antibody was formulated.

| Buffers | pH |
|---|---|
| 20 mM acetate | 4, 5, 5.5, 6 |
| 20 mM acetate with NaCl (75 mM or 150 mM) | 5, 5.5 |
| 20 mM acetate with sucrose (3.5 or 7%) | 5, 5.5 |
| 20 mM phosphate | 5, 6, 7, 8, 9 |
| 20 mM phosphate with NaCl (75 mM or 150 mM) | 5 |
| 20 mM phosphate with sucrose (3.5 or 7%) | 5 |

Methods.
Structural Studies

Structural studies were carried out by using circular dichroism (CD). Secondary and tertiary structures were studied by using far UV circular dichroism (FUV) and near UV circular dichroism (NUV) respectively.

Thermal Denaturation Studies

Protein structural changes were monitored using differential scanning calorimetry (DSC), far UV-circular dichroism spectroscopy (FUV CD), near UV-circular dichroism spectroscopy (NUV CD), tryptophan fluorescence spectroscopy (TRP FL), and particle size by light scattering (PS) as the samples were heated at a constant rate.

Short Term Stability Studies

Real time stability of the antibody was studied in 20 mM acetate buffer pH 5.5 with sucrose. The stability conditions used were 4, 25 and 40° C. and the samples were kept for 1 month. The percentage monomer content was analyzed by using HPSEC assay.

Results and Discussion

Far UV (FUV) circular dichroism scan in acetate buffer at pH5. A minimum of 217 nm and shoulder at 235 nm indicate the predominant presence of β-sheet secondary structure. Maximum at 202 nm is due to presence of β-turn secondary structures (see FIG. 1(a)).

Near UV (NUV) circular dichroism scan in acetate buffer at pH5. Near UV CD spectrum shows three distinct regions:
250-270 nm: phenylalanine residues,
270-290 nm: tyrosine residues,
280-300 nm: tryptophan residues (see FIG. 1(b)).

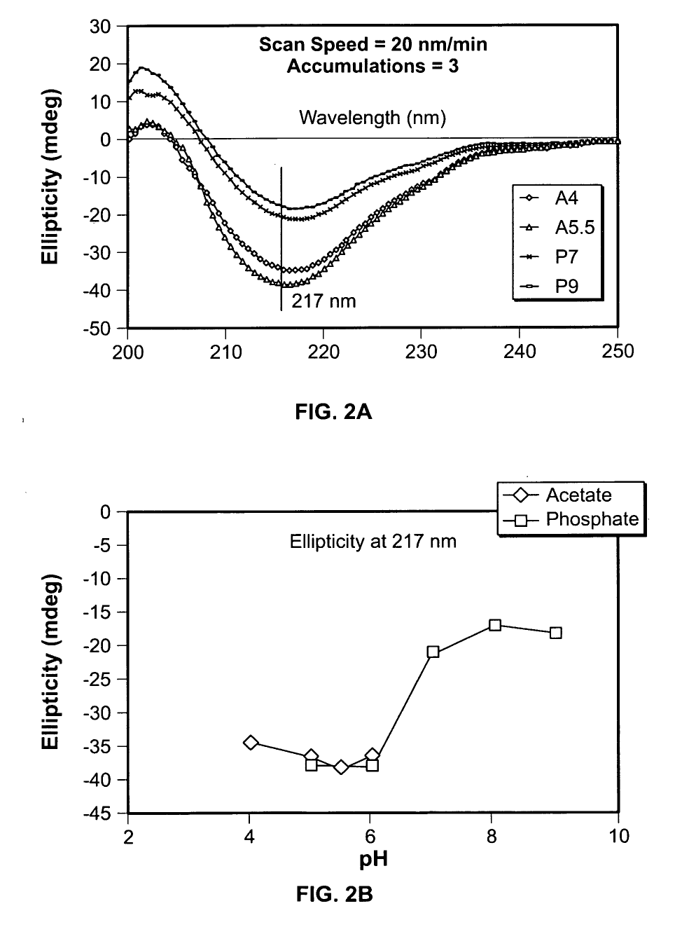

Far UV (FUV) circular dichroism scan in various buffers. As shown in FIG. 2(a), change in ellipticity with pH was observed at 217 nm, 235 nm and 202 nm. The minimum values of ellipticity corresponding to β-sheet secondary structure was observed between pH 5 and 6.

Changes in ellipticity as a function of pH. For pH above 6, ellipticity increases signifying structural change in β-sheet secondary structure (FIG. 2(b)). Similar trend was observed at 235 nm (FIG. 2(c)). Ellipticity at 202 nm increases above pH 6, which suggests an increase in β-turn secondary structure (FIG. 2(d)).

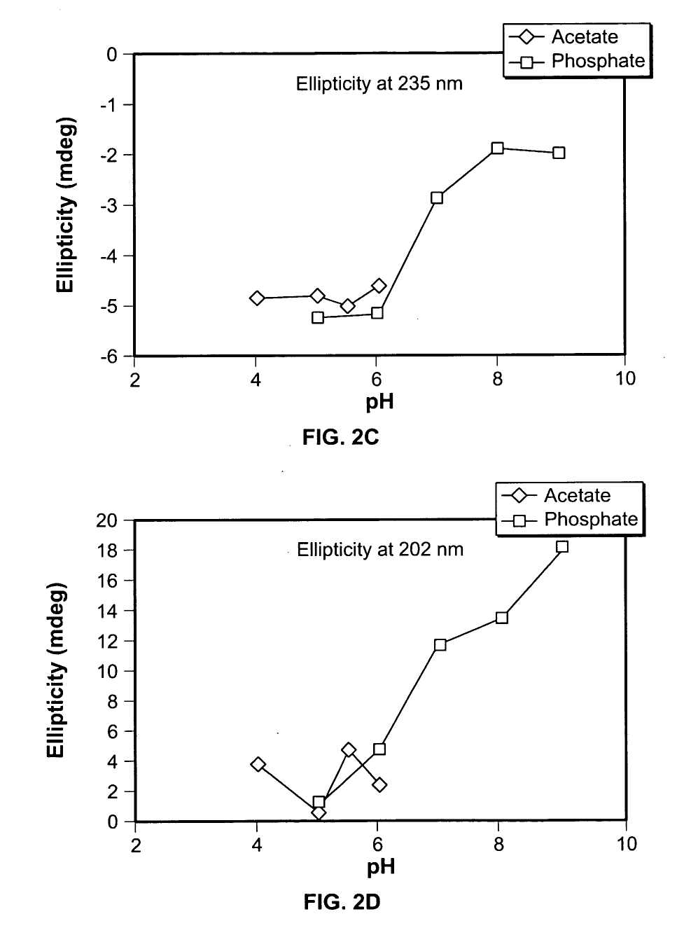

Near UV (FUV) circular dichroism scan in various buffers. No appreciable change in tertiary structure was observed (see FIG. 3).

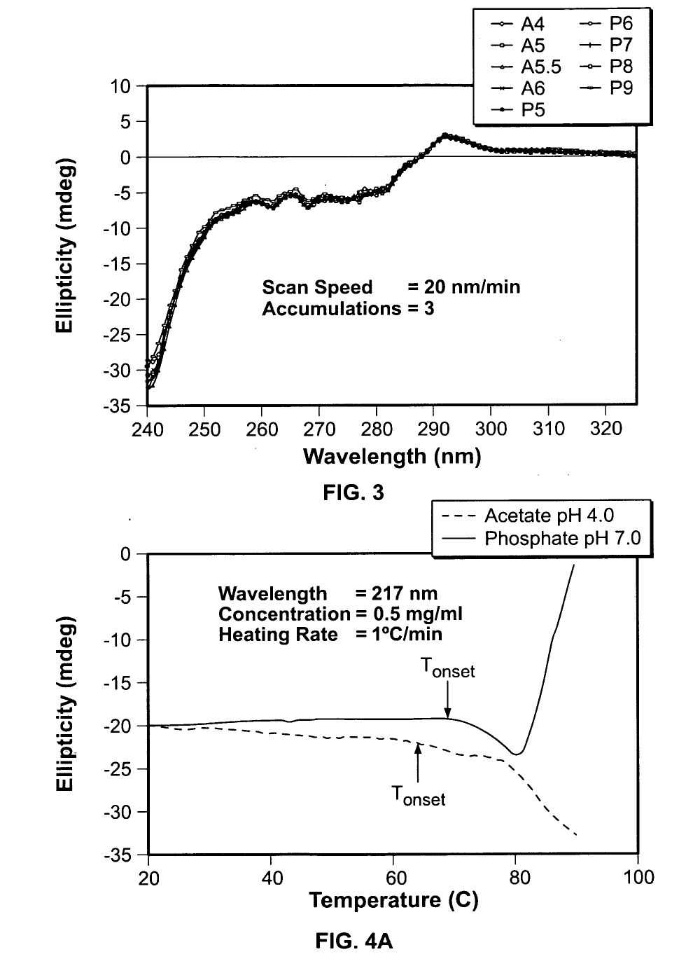

Thermal studies. On heating samples from 20-63° C. no change was seen in the CD signal of the anti-IGF1R antibody signifying no change in the secondary structure in either buffer. At Tₒₙₛₑₜ (64.1° C., pH 4) a decrease in CD signal was seen due to unfolding and change in secondary structure. The ellipticity further increased with increase in temperature possibly due to formation of intermolecular β-sheet secondary structure in aggregates. The anti-IGF1R antibody in phosphate buffer at pH 7 showed Tₒₙₛₑₜ at 68.3° C. At 80° C., an decrease in ellipticity was observed possibly due to precipitation and loss of the anti-IGF1R antibody in solution. Acetate buffer at pH 5.5 depicted highest onset temperature compared to other buffers. See FIG. 4.

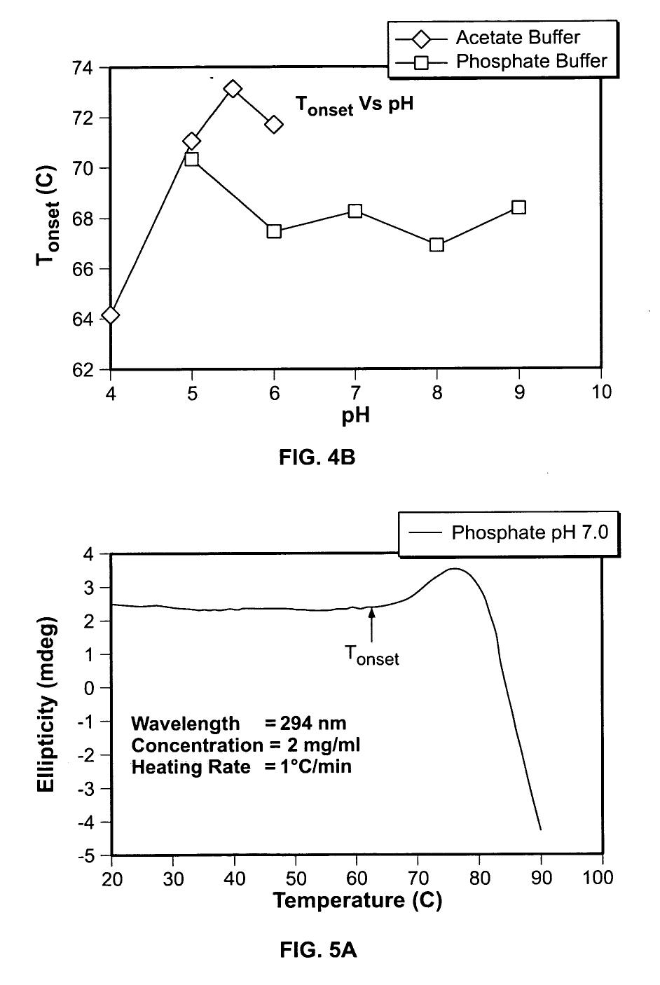

On heating the anti-IGF1R antibody samples from 20-60° C., ellipticity by NUV CD remained constant at 294 nm (FIG. 5(a)). At 61° C., an increase in the ellipticity can be seen which was followed by a decrease in ellipticity suggesting local changes in tryptophan environment due to unfolding of protein. Tₒₙₛₑₜ temperatures for acetate buffer at pH 5.5 and 6 were higher than that seen for other buffers (FIG. 5(b)).

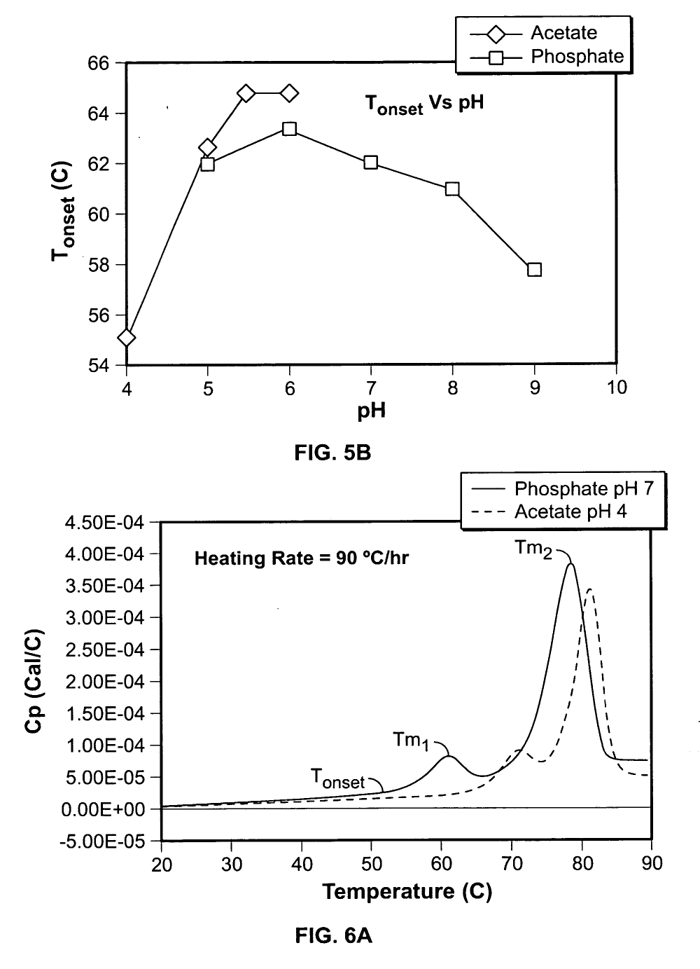

DSC thermograms showed two transition temperatures, Tₘ₁ and Tₘ₂ (FIG. 6(a)). These are the temperatures at which maximum enthalpy change occurs due to protein structural change. Highest Tₒₙₛₑₜ temperature was observed in acetate buffer at pH 5.5 (FIG. 6(b)). Acetate buffer at pH 6 showed highest Tₘ₁ at 69.9° C. (FIG. 6(c)) while acetate buffer at pH 5.5 and 6.0 depicted highest Tₘ₂ at 82.2 and 82.3° C. respectively (not shown).

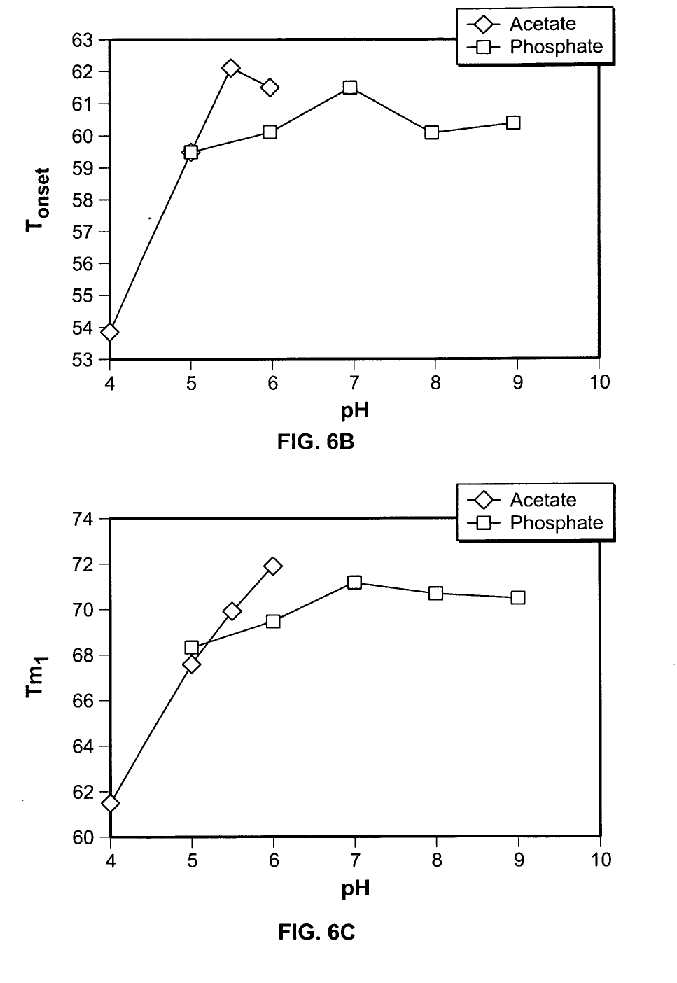

Particle size/aggregation studies. FIG. 7(a) shows particle size distribution obtained for the anti-IGF1R antibody. Mean size of anti-IGF1R antibody in all the buffers tested was 11.05 nm. FIG. 7(b) shows the change in size distribution of anti-IGF1R antibody at various temperatures. As temperature increases, increase in size can be observed due to aggregate formation.

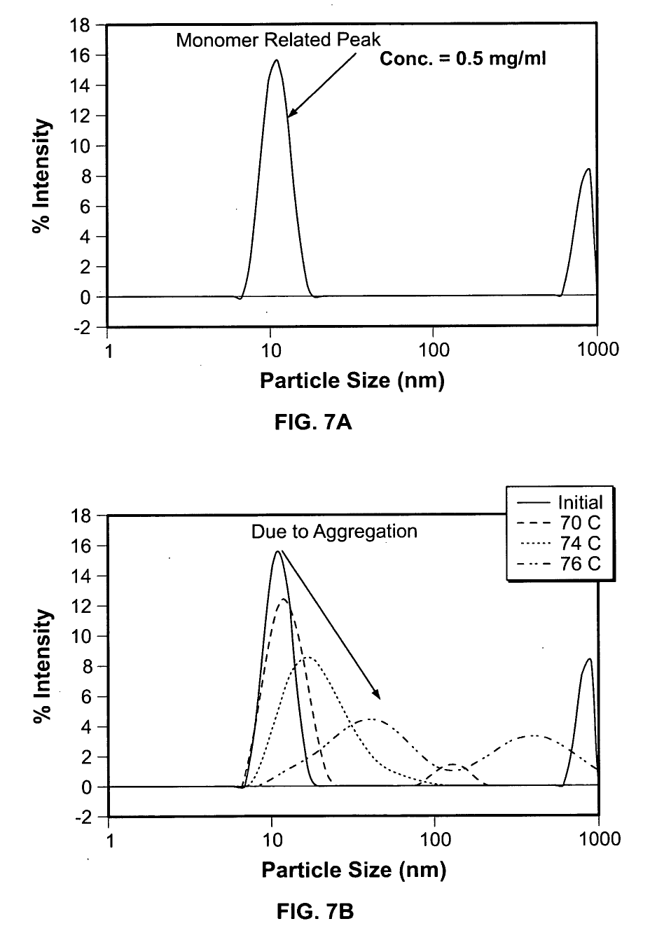

Phosphate buffer at pH 5 showed highest Tₒₙₛₑₜ of aggregation at 76° C. Acetate buffers at pH 5, 5.5 and 6 showed Tₒₙₛₑₜ of aggregation at 74° C. while remaining buffers showed aggregation at 70° C. (see FIG. 8(a)). Tₒₙₛₑₜ of aggregation was not observed in acetate buffer at pH 4 (see FIG. 8(b)).

TABLE 3
Summary of thermal melt data obtained by various techniques.

| Buffer | TRP FL | FUV CD | NUV CD | DSC | | | PS |
|---|---|---|---|---|---|---|---|
| Solution | Tₒₙₛₑₜ | Tₒₙₛₑₜ | Tₒₙₛₑₜ | Tₒₙₛₑₜ | Tₘ₁ | Tₘ₂ | Tₘ |
| Ace 4.0 | 63.9 | 64.1 | 55.0 | 53.8 | 61.4 | 78.8 | — |
| Ace 5.0 | 64.9 | 71.1 | 62.7 | 59.6 | 67.6 | 81.1 | 74.0 |
| Ace 5.5 | 68.4 | 73.2 | 64.8 | 62.2 | 69.9 | 82.2 | 74.0 |
| Ace 6.0 | 62.9 | 71.8 | 64.8 | 61.6 | 71.9 | 82.3 | 74.0 |
| Phos 5.0 | 60.4 | 70.4 | 62.0 | 59.5 | 61.3 | 81.8 | 76.0 |
| Phos 6.0 | 61.4 | 67.6 | 63.4 | 60.2 | 69.4 | 82.2 | 74.0 |
| Phos 7.0 | 61.9 | 68.3 | 62.0 | 61.5 | 71.2 | 81.5 | 70.0 |
| Phos 8.0 | 60.9 | 66.9 | 61.0 | 60.1 | 70.7 | 80.8 | 70.0 |
| Phos 9.0 | 60.0 | 68.3 | 57.6 | 60.4 | 70.4 | 80.7 | 70.0 |

The anti-IGF1R antibody exhibited higher Tₒₙₛₑₜ and Tₘ in the pH region of 5 and 6. Most techniques showed higher Tₒₙₛₑₜ and Tₘ in acetate buffer at pH 5.5.

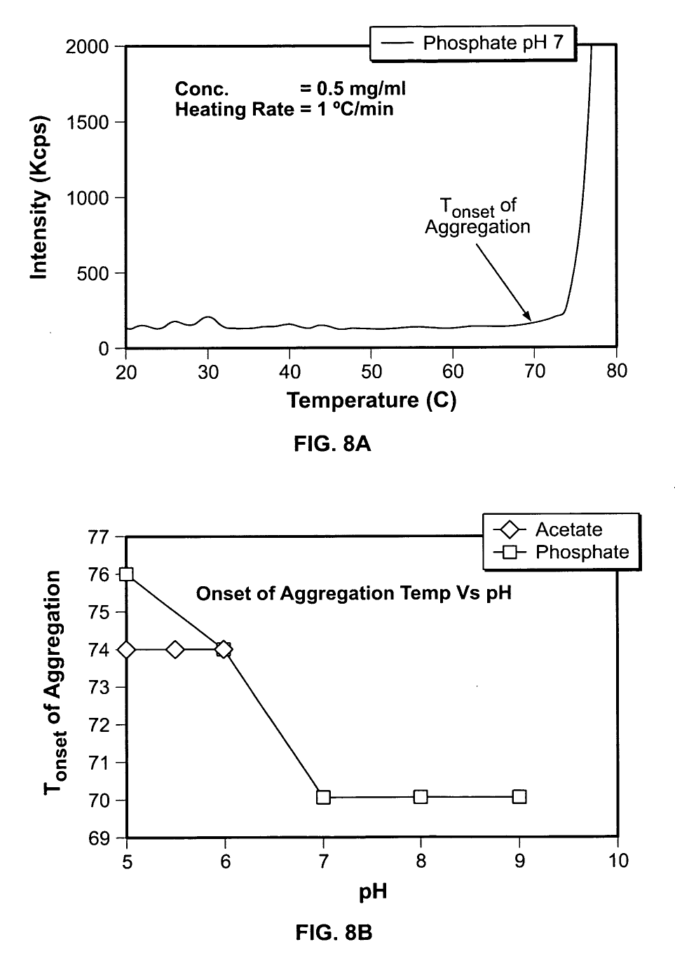

Effect of NaCl or sucrose on Tₒₙₛₑₜ. The addition of sodium chloride decreased FUV CD Tₒₙₛₑₜ temperatures indicating that protein unfolding occurs at lower temperature. Similar trends were seen when the effect of sodium chloride on the anti-IGF1R antibody was studied using NUV CD, TRP FL, PS and DSC. See FIG. 9.

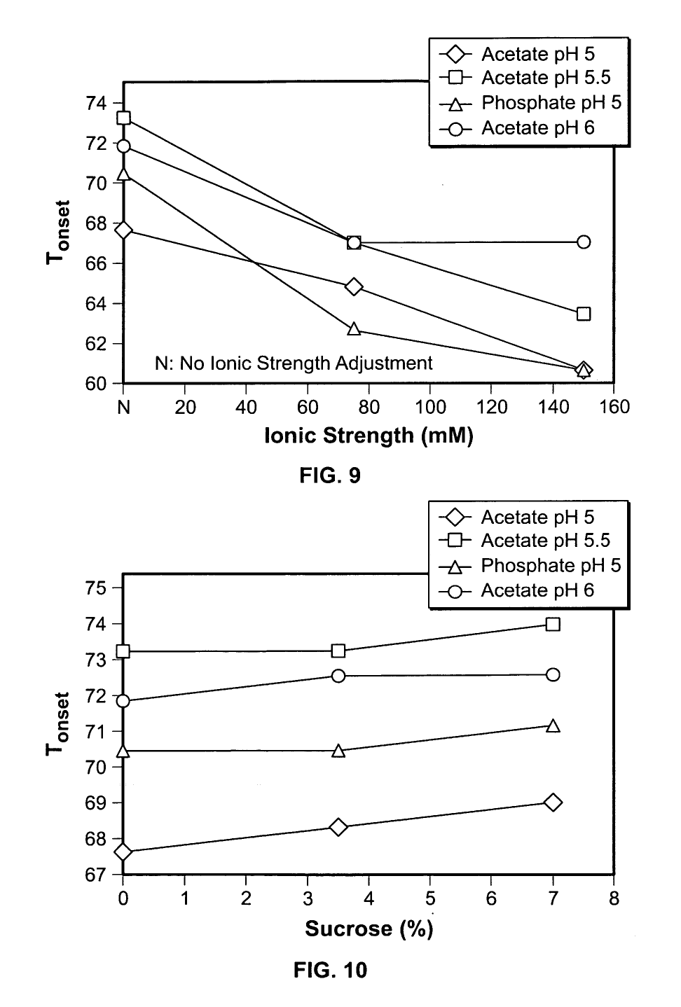

The addition of sucrose increased FUV CD Tₒₙₛₑₜ temperatures indicating that protein unfolding occurs at higher temperature. Similar trends were seen when the effect of sucrose on the anti-IGF1R antibody was studied using NUV CD, TRP FL, PS and DSC. See FIG. 10.

These experiments demonstrated that sucrose had a stabilizing effect on the anti-IGF1R antibody.

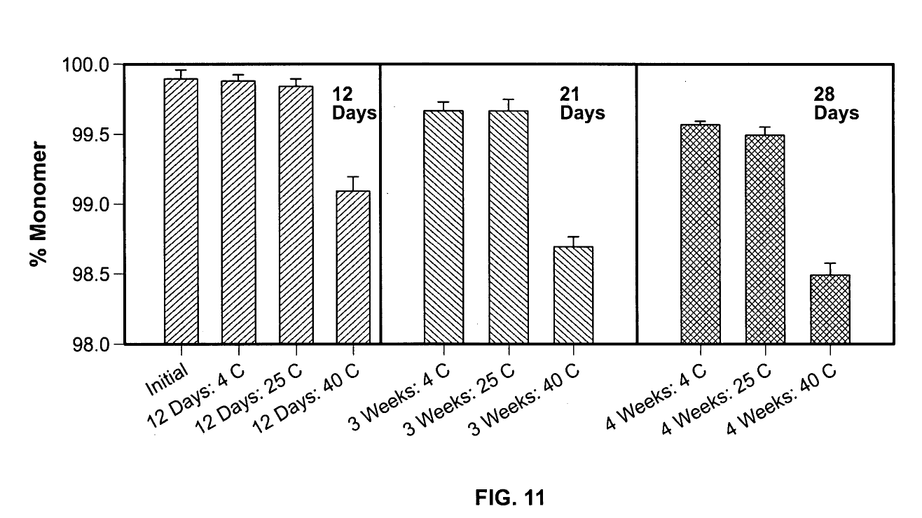

Stability study of the anti-IGFlR antibody in acetate buffer, 7% sucrose and pH5.5. The anti-IGF1R antibody (15 mg/ml) in 20 mM acetate buffer at pH 5.5 with 7% w/v sucrose was placed on stability at 4° C., 25° C. and 40° C. After 12 days, the monomer content for 40° C. decreased to 99%. The monomer content at 4° C. and 25° C. were comparable to initial. After 21 and 28 days, monomer content for 40° C. sample decreased to 98.7% and 98.5%, respectively. At 4 and 25° C., monomer content dropped slightly (approximately 0.2%) compared to initial. See FIG. 11.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A pharmaceutical formulation comprising an isolated antibody or antigen-binding fragment thereof that binds specifically to IGF1R, a buffer and sucrose.

2. The formulation of claim 1 wherein the buffer is phosphate buffer, citrate buffer, histidine buffer, glycine buffer or acetate buffer.

3. The pharmaceutical formulation of claim 1 comprising an antibody or antigen-binding fragment thereof that binds specifically to IGF1R, a buffer and sucrose at a pH of about 5.5 to about 6.0.

4. The formulation of claim 1 wherein the buffer is phosphate buffer, citrate buffer, histidine buffer, glycine buffer or acetate buffer.

5. The formulation of claim 1 wherein the antibody or fragment comprises one or more light chain complementarity determining regions selected from the group consisting of SEQ ID NOs: 1-3; and/or one or more heavy chain complementarity determining regions selected from the group consisting of SEQ ID NOs: 4-7.

6. The pharmaceutical formulation of claim 1 comprising an antibody or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; a buffer and sucrose at a pH of about 5.5 to about 6.0.

7. The formulation of claim 1 which is lyophilized.

8. The formulation of claim 1 which is sterile.

9. The formulation of claim 1 wherein the antibody or fragment comprises a heavy chain constant region selected from the group consisting of γ1, γ2, γ3 and γ4 or a κ light chain constant region.

10. The formulation of claim 1 in an aqueous solution.

11. The formulation of claim 1 wherein the antibody or fragment concentration is about 20 mg/ml.

12. The formulation of claim 1 wherein the concentration of buffer is about 1 to about 20 mM.

13. The formulation of claim 1 wherein the concentration of sucrose is about 5 to about 70 mg/ml.

14. The formulation of claim 1 in association with a further therapeutic agent.

15. The formulation of claim 1 wherein the further therapeutic agent is one or more members selected from the group consisting of:

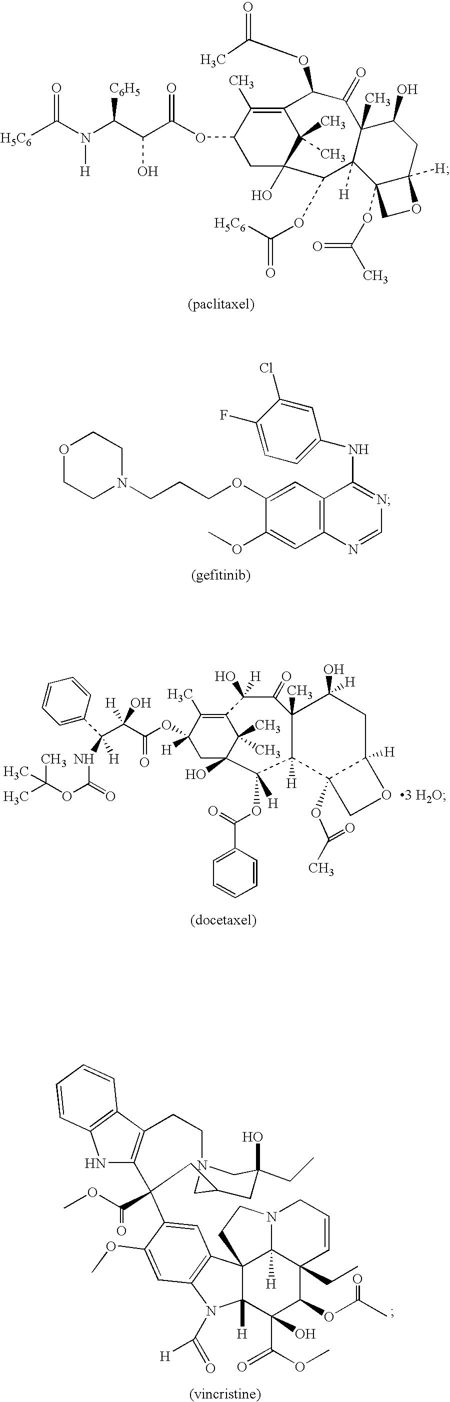

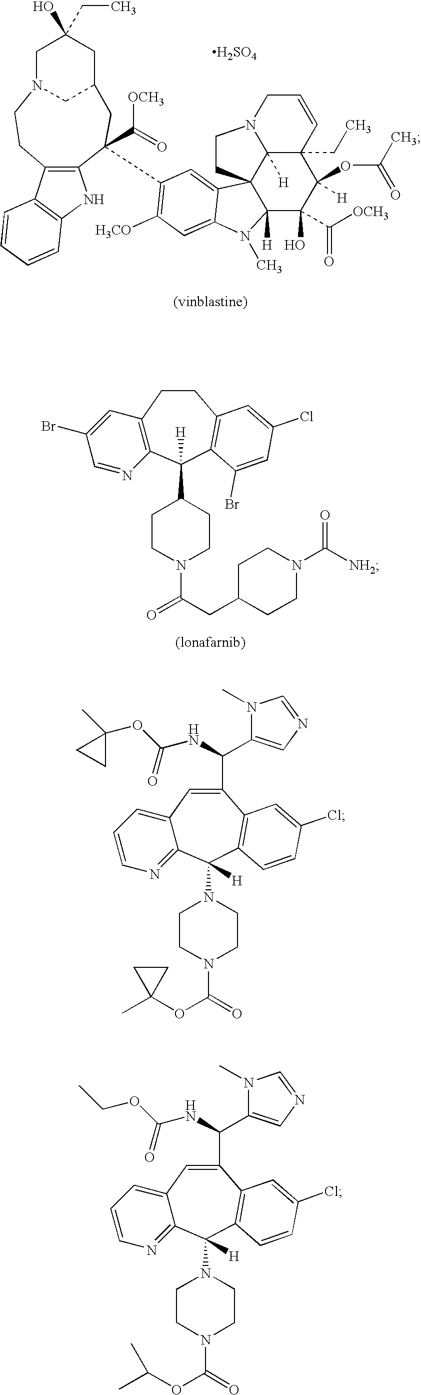

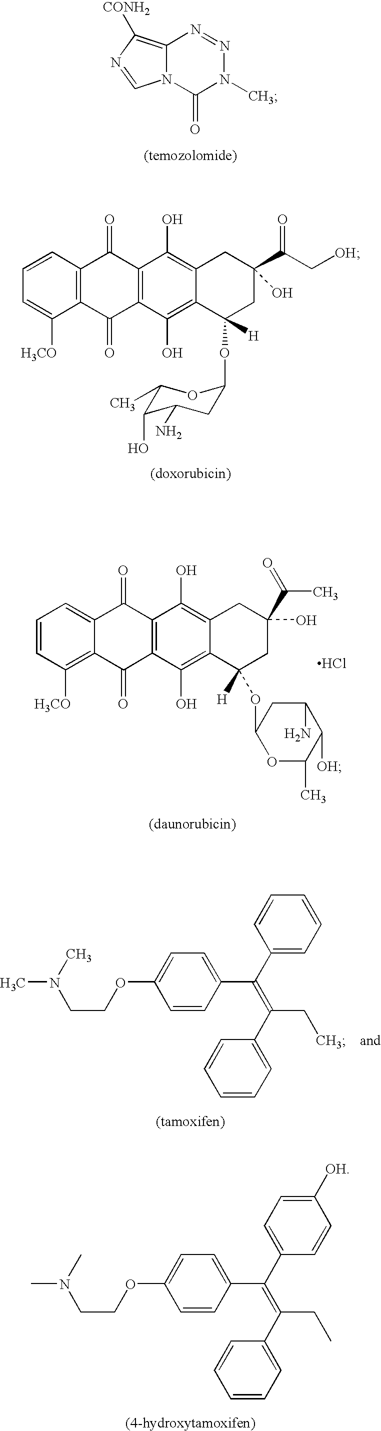

16. The formulation of claim 1 comprising, in a single composition, the antibody or fragment comprising a light chain variable region selected from the group consisting of amino acids 20-128 of SEQ ID NOs: 8-14 and/or a heavy chain variable region selected from the group consisting of amino acids 20-137 of SEQ ID NOs: 15-17; acetate; acetic acid and sucrose at a pH of about 5.5 in association with a further therapeutic agent.

17. The pharmaceutical formulation of claim 1, at a pH of 5.5, comprising:
(a) 20 mg/ml of an antibody or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17;
(b) 2.3 mg/ml of sodium acetate trihydrate;
(c) 0.18 mg/ml of glacial acetic acid;
(d) 70 mg/ml of Sucrose; and
(e) water.

18. A lyophilized pharmaceutical formulation, at a pH of 5.5, which, when reconstituted comprises:
(a) 20 mg/ml of a therapeutically effective amount of an antibody or antigen-binding fragment thereof comprising a light chain variable region selected from the group consisting of amino acids 20-128 and/or a heavy chain variable region selected from the group consisting of amino acids 20-137 of SEQ ID NOs: 15-17;
(b) 2.3 mg/ml of sodium acetate trihydrate;
(c) 0.18 mg/ml of glacial acetic acid; and
(d) 70 mg/ml of Sucrose; and
(e) water.

19. A vessel comprising the formulation of claim 1.

20. The vessel of claim 19 which is a glass vial.

21. An injection device comprising the formulation of claim 1.

22. The injection device of claim 21 which is a hypodermic needle and syringe.

23. A kit comprising
(a) the formulation of claim 1 in a vessel or injection device; and
(b) a package insert comprising one or more items of information regarding said formulation selected from the group consisting of pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

24. A method for treating or preventing a medical disorder mediated by IGF1R, IGF-1 and/or IGF-2, in a subject, comprising administering, to the subject, a therapeutically effective amount of a formulation of claim 1.

25. The method of claim 24 wherein the medical disorder is selected from the group consisting of neuroblastoma, rhabdomyosarcoma, osteosarcoma, pediatric cancer, acromegaly, ovarian cancer, pancreatic cancer, benign prostatic hyperplasia, breast cancer, prostate cancer, bone cancer, lung cancer, colorectal cancer, cervical cancer, synovial sarcoma, bladder cancer, gastric cancer, Wilm's cancer, ovarian cancer, benign prostatic hyperplasia (BPH), diarrhea associated with metastatic carcinoid and vasoactive intestinal peptide secreting tumors , VIPoma, Werner-Morrison syndrome, kidney cancer, renal cell carcinoma, transitional cell cancer, Ewing Sarcoma, leukemia, acute lymphoblastic leukemia, brain cancer, glioblastoma, non-glioblastoma brain cancer, meningioma, pituitary adenoma, vestibular schwannoma, a primitive neuroectodermal tumor, medulloblastoma, astrocytoma, oligodendroglioma, ependymoma, choroid plexus papilloma, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

26. The method of claim 24 wherein the subject is administered a further therapeutic agent in association with the formulation.

27. The method of claim 26 wherein the further therapeutic agent is selected from the group consisting of:

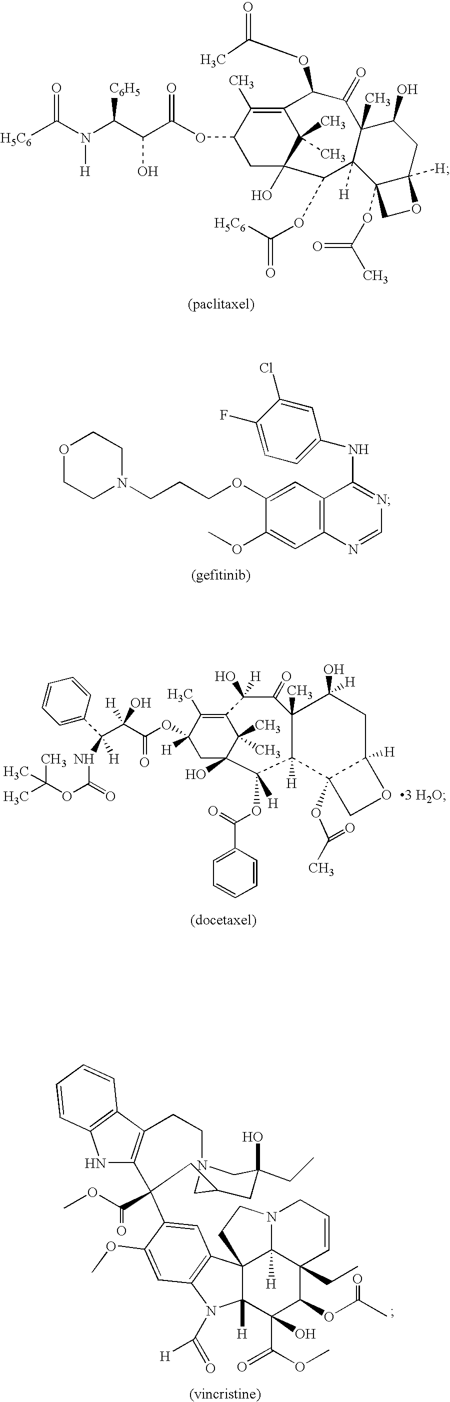

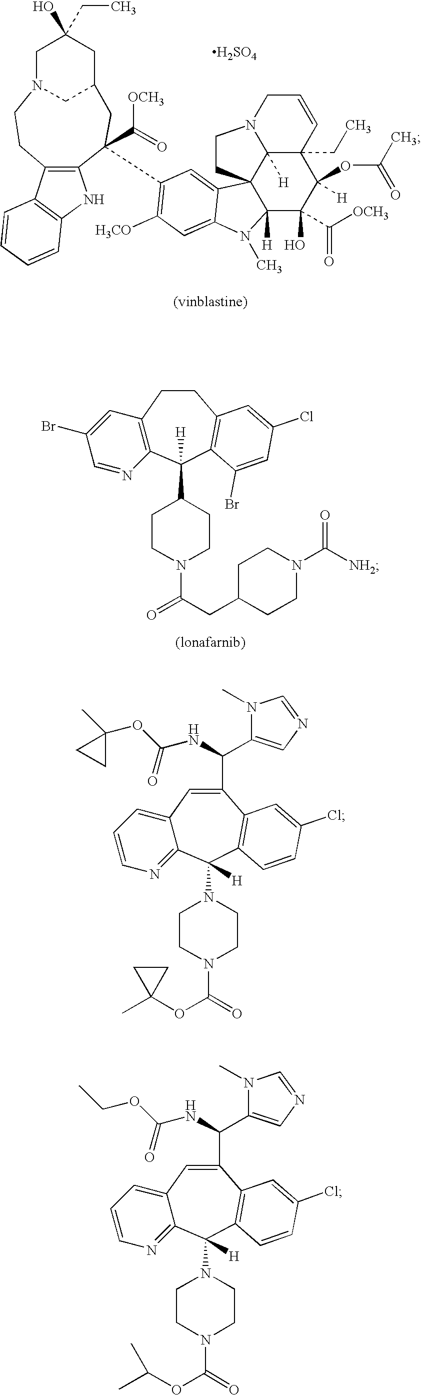

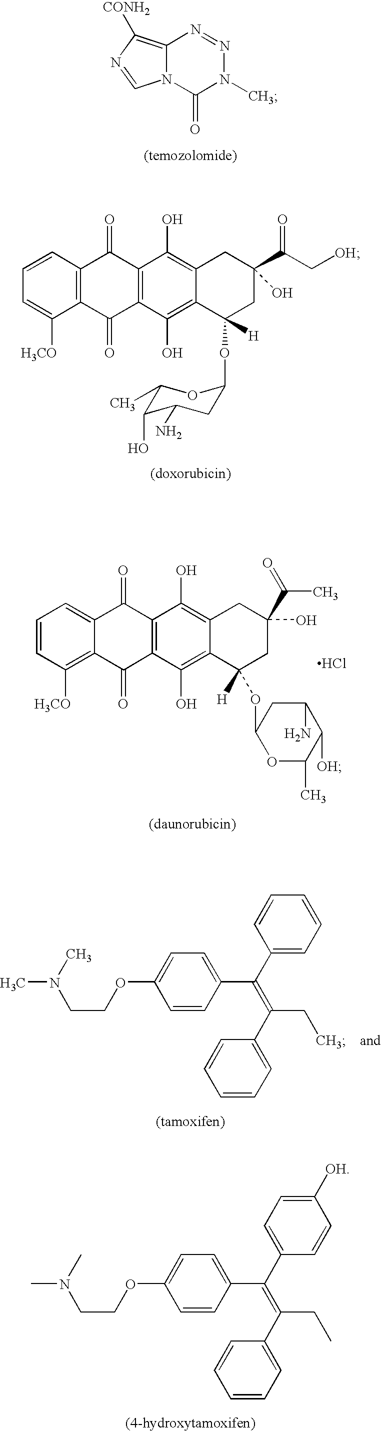

28. The method of claim 24 wherein the subject is a human.

29. The method of claim 24 wherein the formulation is administered to the subject parenterally.

30. The method of claim 24 wherein the formulation is at about pH 5.5 and comprises:
(a) 20 mg/ml of a therapeutically effective amount of an antibody or antigen-binding fragment thereof comprising a light chain variable region selected from the group consisting of amino acids 20-128 of SEQ ID NOs: 8-14 and/or a heavy chain variable region selected from the group consisting of amino acids 20-137 of SEQ ID NOs: 15-17;
(b) 2.3 mg/ml of sodium acetate trihydrate;
(c) 0.18 mg/ml of glacial acetic acid;
(d) 70 mg/ml of Sucrose; and
(e) water.

31. A method for stabilizing an antibody or antigen-binding fragment thereof comprising a light chain variable region selected from the group consisting of amino acids 20-128 of SEQ ID NOs: 20-128 and/or a heavy chain variable region selected from the group consisting of amino acids 20-137 of SEQ ID NOs: 15-17; comprising combining said antibody or fragment with acetate; acetic acid and sucrose, optionally, at a pH of about 5.5.

32. The method of claim 31 wherein the antibody or fragment concentration is about 20 mg/ml.

33. The method of claim 31 wherein the concentration of acetate is about 2.3 mg/ml, the concentration of acetic acid is about 0.18 mg/ml and the concentration of sucrose is about 70 mg/ml.
